(19) United States
(12) Patent Application Publication
(10) Pub. No.: US 2016/0058698 A1
(43) Pub. Date: Mar. 3, 2016

(54) COMPOSITION FOR CONTROLLED DELIVERY OF BIOACTIVE AGENTS

(71) Applicant: COMMONWEALTH SCIENTIFIC AND INDUSTRIAL RESEARCH ORGANISATION, Campbell, Australian Capital Territory (AU)

(72) Inventors: Roshan Tyrrel Anton MAYADUNNE, Wheelers Hill, Victoria (AU); Palak Hardik BHATT, Lyndhurst, Victoria (AU); Ashita Indaka BALACHANDRA, Ringwood, Victoria (AU); Thi Phuong Tam LE, Clematis, Victoria (AU)

(73) Assignee: COMMONWEALTH SCIENTIFIC AND INDUSTRIAL RESEARCH ORGANISATION, Campbell, Australian Capital Territory (AU)

(21) Appl. No.: 14/784,217

(22) PCT Filed: Apr. 11, 2014
(86) PCT No.: PCT/AU2014/000400

§ 371 Date: Oct. 13, 2015

(30) Foreign Application Priority Data
Apr. 11, 2013 (AU) 2013901241

(51) Int. Cl. A61K 9/00; A61K 47/34

(52) U.S. Cl. CPC A61K 9/0024; A61K 47/34

(57) ABSTRACT

The present invention relates to an injectable composition for controlled delivery of a bioactive agent. The injectable composition is capable of spontaneous gelation in situ upon administration to a subject to form a gel from which an effective amount of the bioactive agent can be released over a period of 7 days or more.

### FIELD OF THE INVENTION

The present invention relates to injectable compositions for the controlled delivery of bioactive agents. The injectable composition comprises a biodegradable non-linear polymer and is capable of gelation at physiological temperature, to form a gelled composition that provides for controlled release of a bioactive agent over a sustained period of time.

### BACKGROUND

Polymer gels may fall into the categories of chemical gels or physical gels. In chemical gels, network formation or gelation of a polymer involves the formation of covalent bonds or crosslinks between polymer chains. The crosslinking of polymer chains via covalent bonds in such cases is irreversible due to the nature of bonding and hence the reversion of gel (semi-solid or solid) to sol (or liquid or flowing material) is not possible. In contrast to chemical gels, physical gels are formed via the physical association of polymer chains, initially though the formation of micelles at critical micellar concentrations, and subsequent network formation through micelle aggregation into a percolated “micelle-network”. The temperature at which the sol to gel transformation occurs in physical gels is referred to as the critical gelation temperature. Physical gels that exhibit reverse gelation behaviour in response to heat may be referred to as thermo-reversible gels and in the case of polymers they are referred to as thermoreversible polymers (TRPs). Many natural and synthetic TRPs have been described and their thermoresponsive behaviour investigated (Jeong et al, Advanced Drug Delivery reviews, Vol 54, 2002, 37-51). Many of the synthetic TRPs are based on linear block copolymers that are required to have defined quantities of polymer blocks of defined composition in order to achieve reversible thermoresponsive gelation behaviour.

Of these, many are based on poly(N-isopropyl acrylamide) (poly(NIPAM)) or polyether block copolymers of ethylene oxide and propylene oxide (poloxomers or Pluronic®), or poly(ethylene glycol)-b-poly(propylene glycol)-b-poly(ethylene glycol) (PEO—PPO-PEO) triblocks. TRPs based on poly(NIPAM) have been found to be unacceptable for biomedical applications due to their toxicity in vivo. TRPs based on derivatives of the polyether block copolymers may be acceptable in biomedical applications, depending on the molecular weight of the copolymer. However, a major drawback of both the poly(NIPAM) and poloxamer classes of polymers are their relative non-degradability in a biological environment. The low mechanical strength of gels formed with poloxomers as a result of molecular weight restrictions is another issue that may limit their use.

Attempts have been made to develop biodegradable thermoreversible polymers, specifically for drug delivery applications. However, a number of biodegradable TRPs suffer from a lack of consistency in the gel matrices formed, have poor shape retention following implantation or a lack of stability in vivo, or can only provide drug release over a relatively short period of time.

A proprietary linear ABA block copolymer known as ReGel®, is a biodegradable TRP composed of a poly(lactic/glycolic acid)-block-poly(ethylene glycol)-block-poly(lactic/glycolic acid) (PLGA-PEG-PLGA) copolymer that has been reported to be effective in drug delivery. However, a limitation with ReGel® is its relatively short drug delivery time. For example, it has been reported that hydrophilic bioactive agents such as enzymes and proteins are completely released from ReGel® in about 15 days (J. Controlled Release, Vol 172, pg 203, 2001, J. Immunology, Vol. 29, pg 524, 2006). Other limitations with ReGel® include significant diffusion controlled burst release of the drug within the first 48 hours following administration, and rapid in vivo degradation of the polymer leading to rapid drug release, which results in an unfavourable decrease in pH in the immediate vicinity of the administration site due to an increase in acidic PLGA fragments.

It would be desirable to provide an injectable composition for controlled delivery of a bioactive agent that addresses or at least ameliorates one or more disadvantages of the prior art.

The discussion of documents, acts, materials, devices, articles and the like is included in this specification solely for the purpose of providing a context for the present invention. It is not suggested or represented that any or all of these matters formed part of the prior art base or were common general knowledge in the field relevant to the present invention as it existed before the priority date of each claim of this application.

### SUMMARY

The present invention provides an injectable composition for controlled delivery of a bioactive agent. The injectable composition is capable of gelation to form an implant in situ after injection. The implant is biodegradable and the bioactive agent can be released from the implant over a period of 7 days or more, providing for a sustained bioactive effect.

In one aspect the present invention provides an injectable composition for controlled delivery of a bioactive agent comprising:
a biodegradable, thermoreversible, non-linear block copolymer;
an aqueous solvent; and
a bioactive agent,

wherein the composition is injectable as a liquid and the liquid is converted to a gel at physiological temperature, and wherein the gelled composition provides release of an effective amount of the bioactive agent over a period of at least 7 days.

In one set of embodiments the gelled composition provides release of an effective amount of the bioactive agent over a period of at least 14 days, preferably at least 28 days.

The injectable composition comprises a biodegradable thermoreversible, non-linear block copolymer. The non-linear block copolymer may have an architecture selected from the group consisting of a branched, hyperbranched, comb, brush, star and dendritic. In one set of embodiments the non-linear block copolymer is a biodegradable star block copolymer.

The biodegradable, thermoreversible, star block copolymer comprises a plurality of polymer arms attached to and extending from a multi-valent central core. In specific embodiments, the polymer arms each comprise a block copolymer. One or more components of the star block copolymer (e.g. the core and/or the arms of the star copolymer) is biodegradable.

The biodegradable, thermoreversible star block copolymer may be of formula A(BC)ₙ, wherein A represents an n-valent core and one of B and C represents a hydrophobic block and the other of B and C represents a hydrophilic block and n is an integer and is at least 3. In some embodiments, n is an integer in the range of from 4 to 8.

The weight ratio of B to C is preferably in the range of from 10:1 to 1:10. In some embodiments, the weight ratio of B to C is in the range of from 6:1 to about 1:6, or from about 3:1 to about 1:3.

The hydrophobic block present in the biodegradable, thermoreversible non-linear block copolymer may have a molecular weight in the range of from about 500 to about 15,000, preferably from about 1000 to about 10,000, more preferably from about 1500 to about 5000.

The hydrophilic block present in the biodegradable, thermoreversible non-linear block copolymer may have a molecular weight in the range of from about 100 to about 3000, preferably from about 250 to about 2000, more preferably from about 200 to about 1500.

In one set of embodiments the biodegradable, thermoreversible, non-linear block copolymer is a biodegradable star block copolymer of formula A(BC)ₙ, where B represents a hydrophobic block and C represents a hydrophilic block. For example, B may comprise a biodegradable polyester and C may comprise a polyether.

Biodegradable polyesters present in the non-linear block copolymer may be formed from the polymerisation at least one monomer selected from the group consisting of D,L-lactic acid, D-lactic acid, L-lactic acid, glycolic acid, 8-caprolactone, ε-hydroxy hexanoic acid, γ-butyrolactone, γ-hydroxy butyric acid, 6-valerolactone, 6-hydroxy valeric acid, hydroxy butyric acids, malic acid, mandelic acid and mixtures thereof.

In one embodiment the biodegradable polyester is obtained from the polymerisation of D,L-lactic acid and ε-caprolactone. The biodegradable polyester may therefore be a copolymer of D,L-lactic acid and ε-caprolactone. In such embodiments, the mole ratio of D,L-lactic acid to s-caprolactone in the biodegradable polyester may be in the range of from 90:10 to 10:90, preferably from 75:25 to 25:75.

Polyethers that may be present in the biodegradable, thermoreversible, non-linear block copolymer may be selected from poly(ethylene glycol), poly(propylene glycol), and copolymers thereof.

In star block copolymers of formula A(BC)ₙ, the blocks B and C may be covalently coupled via a linking group. In one form, the linking group is derived from a diisocyanate.

In some embodiments the star block copolymer may comprise a further block (D). When present, the further block (D) will generally be in an arm of the star block copolymer.

In one set of embodiments when D is present, the star block copolymer may be of formula A(BCD)ₙ.

The injectable composition may comprise no more than about 50% (w/w), preferably no more than 30% (w/w), of non-linear block copolymer.

The injectable composition is an aqueous composition and may comprise at least 50% (w/w), preferably at least 60% (w/w) and more preferably at least 70% (w/w) of aqueous solvent. The aqueous solvent is preferably water.

In one set of embodiments the injectable composition has a storage modulus in the range of 1 to 20 Pa when in liquid form and when in gel form at physiological temperature.

The injectable composition comprises a bioactive agent that is capable of exerting a therapeutic or prophylactic effect in a subject. The bioactive agent may be selected from the group consisting of hydrophilic drugs, hydrophobic drugs, proteins and antibodies, hormones, genes or nucleic acids, oligonucleotides, actives for antisense therapy, polysaccharides and other sugars, lipids, gangliosides, vasoactive agents, neuroactive agents, anticoagulents, immunomodulating agents, anti-cancer agents, anti-inflammatory agents, antibiotics antivirals, vaccines, and combinations thereof.

The injectable composition may comprise an amount of bioactive agent in an amount in the range of from about 0.01% to about 20% by weight of the composition.

In some embodiments, the injectable composition further comprises an additive that enhances control of the release of the bioactive agent. In one set of embodiments the additive is a polysaccharide. When present, the polysaccharide may be selected from the group consisting of xanthan gum, welan gum, dextran, gellan, pullulan, guar gum, locust beam gum, chitin, alginate and mixtures thereof.

The invention enables release of a bioactive agent at a desired site of action to be controlled. In some embodiments release of the bioactive agent occurs in a single phase. In other embodiments, release of the bioactive agent occurs in at least two separate and distinct phases, more preferably in three separate phases. In some embodiments, release of the bioactive agent from the gelled composition is initially burst release followed by diffusion controlled release. In some alternative embodiments, release of the bioactive agent from the gelled composition is initially burst release followed by diffusion controlled release and finally, release under degradation control.

In another aspect the present invention relates to use of an injectable composition according to any one of the embodiments described herein in the manufacture of a medicament for the prophylaxis or treatment of a disease or disorder in a subject.

In a further aspect the present invention relates to a method of treating or preventing a disease or disorder in a subject in need of such treatment or prevention, the method comprising the step of administering an injectable composition of any one of the embodiments described herein into the subject. In one set of embodiments the method comprises injecting the composition from the lumen of a syringe to administer the composition to the subject.

### BRIEF DESCRIPTION OF THE FIGURES

The present invention will now be described with reference to the figures of the accompanying drawings, which illustrate particular embodiments of the present invention, wherein:

FIG. 1 is a schematic illustrating embodiments of non-linear block copolymers that can be employed in the composition of the invention, showing (a) a star shaped block copolymer having arms containing a resorbable hydrophilic block, a biodegradable hydrophobic block and a linking group between the hydrophilic block and the hydrophobic block, and (b) a star shaped block copolymer having a terminal crosslinkable group that facilitates formation of a strong gelled composition.

FIG. 2 is a graph showing the correlation between temperature and modulus for a composition containing the star block copolymer (P(LA₇₅CL₂₅-HDI-PEG(550))₄), in which the storage modulus is increased during gel formation (sol-gel) and the reversibility of the gel back to sol as the temperature is reduced.

FIG. 3 is a graph illustrating the results of a short term release study for a model drug (lysozyme) for a composition comprising a star block polymer (P(LA₇₅CL₂₅-HDI-PEG(550))₄) and comparative compositions comprising prior art linear polymers (Pluronic F127 and ReGel®).

FIG. 4 is a graph illustrating the results of a long term release study for a model drug (lysozyme) for compositions comprising the star bock copolymers P(LA₇₅CL₂₅-HDI-PEG(550))₄, P(LA₇₅CL₂₅-HDI-PEG(350))₄ and a comparative composition comprising prior art linear polymer (Regel®).

FIG. 5 is a graph illustrating the results of a long term release study for small molecular drugs (Drug 1: a platelet inhibitor (dipyridamole): Drug 2 an anticancer drug (doxorubicin) from composition containing the star block copolymer P(LA₇₅CL₂₅-HDI-PEG(550))₄.

FIG. 6 is a photograph showing release study samples demonstrating degradation of the gelled compositions of P(LA₇₅CL₂₅-HDI-PEG(550))₄ from FIG. 5 with dipyridamole and doxorubicin after 13 weeks.

### DETAILED DESCRIPTION

Various terms that will be used throughout the specification have meanings that will be well understood by a skilled addressee. However, for ease of reference some of these terms will now be defined.

The term “bioactive agent” as used herein refers to any chemical or biological material or compound that induces a therapeutic, prophylactic, biological, physiological or pharmacological effect.

The term “physiological temperature” as used herein refers to conditions having a temperature in the range of 25-40 degrees Celsius.

The term “physiological conditions” as used herein refers to conditions having a physiological temperature as defined herein and a pH in the range of 5-8.

The term “biocompatible” as used herein in relation to a substance means the substance is compatible with living tissue. Consequently, the substance is not, or at least is minimally, toxic to living tissue, and does not, or at least minimally and reparably does, injure living tissue; and/or does not, or at least minimally and/or controllably does, cause an immunological reaction in living tissue.

The terms “degradable” and “biodegradable” as used herein in relation to a substance or group (such as a polymer, moiety on a polymer, ligand or linker) means that the substance or group is susceptible to degradation, cleavage or fragmentation over time under physiological conditions or in a biological environment, such as the intracellular environment. Such degradation, cleavage or fragmentation may occur via chemical decomposition (e.g. via hydrolysis or reduction) of suitably labile or degradable moieties under the selected physiological or biological conditions. When used in relation to a polymer substance, the terms “degradable” and “biodegradable” indicate that suitably labile or degradable moieties form part of the molecular structure of the backbone of the polymer. The cleavage or break down of one or more degradable moieties in the polymer backbone leads to fragmentation of the polymer, generally into monomers and/or into lower molecular weight polymer fragments.

The term “injectable” means able to be injected through a surgical needle or catheter for administration subcutaneously, sublingually, buccally, intraocularly, topically, or intramuscularly to a subject. It specifically excludes intravenous administration. Intravenous administration is generally to be avoided due to the risk that the composition could cause blockages to occur in small veins or arteries. Injectable as used herein is also intended to include circumstances in which the composition is thixotropic and is highly viscous or nearly semi solid under static conditions at room temperature but can be converted to a flowable liquid through shear due to the thixotropic properties of the composition, allowing it to become injectable.

The term “effective amount” as used herein means an amount that is sufficient, when administered to a subject suffering from or susceptible to a disease, disorder, and/or condition, to treat, diagnose, prevent, and/or delay the onset of the symptom(s) of the disease, disorder, and/or condition. An effective amount of bioactive agent can be determined by an attending medical practitioner using conventional techniques and by observing results obtained under analogous circumstances. In determining the effective amount a number of factors are to be considered including but not limited to, the species of animal, its size, age and general health, the specific condition involved, the severity of the condition, the response of the patient to treatment, the particular compound administered, the mode of administration, the bioavailability of the preparation administered, the dose regime selected, the use of other medications and other relevant circumstances.

The present invention relates to an injectable composition that is useful in providing controlled delivery of a bioactive agent.

In one aspect, the present invention provides an injectable composition for controlled delivery of a bioactive agent comprising:
a biodegradable, thermoreversible, non-linear block copolymer;
an aqueous solvent; and
a bioactive agent,

wherein the composition is injectable as a liquid and is converted to a gel at physiological temperature and wherein the gelled composition provides release of an effective amount of the bioactive agent over a period of at least 7 days.

The injectable composition of the invention is capable of undergoing gelation to provide a depot from which an effective amount of a bioactive agent can be released over a period of time.

The injectable composition is capable of providing release of an effective amount of a bioactive agent over a period of at least 7 days. The quantity of released bioactive agent that may be considered to be an effective amount may vary according to the type or nature of the bioactive agent and the condition, disease or disorder, or patient or subject to be treated by the bioactive agent.

In some embodiments, the injectable composition is capable of providing release of an effective amount of a bioactive agent over a time period selected from the group consisting of at least 10 days, at least 14 days, at least 21 days, at least 28 days, at least about 35 days, at least about 60 days, and at least about 90 days.

In some embodiments the injectable composition is liquid at room temperature (approximately 20° C.) and converts from a flowable liquid state (sol) to a semi-solid state (gel) as the temperature is increased to physiological temperature (approximately 37° C. in humans).

In other embodiments, the composition of the invention is thixotropic and may be a viscous liquid or nearly semi-solid under static conditions at room temperature (approximately 20° C.). A thixotropic composition is capable of being converted to a flowable liquid state (sol) through injection at the time of administration of the composition. For instance, the composition may exhibit thixotropic behaviour, which allows the viscous composition to become a flowable liquid through shearing as it is injected through the lumen of a needle or the opening in a catheter for example. The flowable liquid subsequently converts to a semi-solid state (gel) as the temperature is increased to physiological temperature (approximately 37° C. in humans).

Upon administration to a desired site in a subject, the injectable composition of the invention solidifies to a gel, with the gel encapsulating the bioactive agent for subsequent controlled release.

The injectable composition of the invention also exhibits thermoreversible behaviour in that the composition can revert from the solid (gel) form to a liquid state (sol) in response to a decrease in temperature.

The injectable composition of the invention comprises a biodegradable, thermoreversible, non-linear block copolymer dispersed in an aqueous medium. The biodegradable, thermoreversible, non-linear block copolymer may have an architecture selected from the group consisting of branched, hyperbranched, comb, brush, star and dendritic. A linear block copolymer of the prior art, as well as examples of some non-linear block copolymer architectures are shown in Scheme 1. Gelation occurs as the copolymer components self-assemble into micelles. The micelles can, in turn, aggregate in response to an increase in temperature, resulting in formation of a gel network.

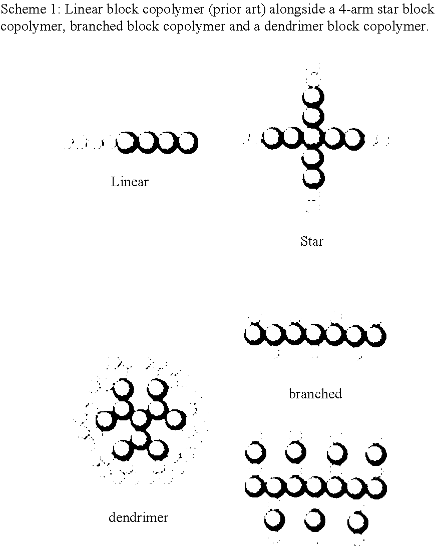

The biodegradable, thermoreversible, non-linear block copolymer comprises at least two different types of repeating subunits or blocks. The different blocks can be derived from at least two different types of monomers, as described below. The use of two or more different blocks to construct the degradable, non-linear block copolymer can advantageously enable the composition, structure and molecular weight of the polymer to be modified and controlled.

Biodegradable, thermoreversible, non-linear block copolymers employed in the injectable composition of the invention comprise at least one hydrophilic block and at least one hydrophobic block. In order for the sol-gel transition to occur in response to temperature, it is necessary that there be an appropriate balance of hydrophilic and hydrophobic blocks in the non-linear block copolymer. The sol-gel transition may be modified by adjusting the ratio of hydrophilic and hydrophobic blocks in the copolymer.

According to one form, the biodegradable, thermoreversible, non-linear block copolymer is a biodegradable star block copolymer. Biodegradable star block copolymers employed in the injectable composition have a plurality of polymer arms extending from a central core. In specific embodiments, the arms of the biodegradable star copolymer each comprise a block copolymer. The block copolymer present in each arm is formed with two or more different types of blocks. Some exemplary block copolymer arms are further discussed below.

In some embodiments, the arms of the biodegradable star block copolymer may each have the same composition. In other embodiments, at least two arms of the biodegradable star block copolymer may be of different composition.

Biodegradable star block copolymers employed in the injectable composition comprise at least 3 arms and in some embodiments, may comprise 4, 6 or 8 arms. Each arm may be composed or comprise a block copolymer, as mentioned above.

In some embodiments the biodegradable star block copolymer may be of formula A(BC)ₙ, wherein A represents an n-valent core, the group (BC) represents an arm of the star block copolymer and n is an integer and is at least 3. In such embodiments, n represents the number of polymer arms present in the star copolymer.

The group (BC) in formula A(BC)ₙ, is a block copolymer arm comprising at least two different blocks. The different blocks are represented by the groups B and C. In one set of embodiments, one of B and C represents a hydrophobic block and the other of B and C represents a hydrophilic block.

In some embodiments, n is an integer selected from the group consisting of 4, 6 and 8. In such embodiments the star block copolymer may comprise 4, 6 or 8 arms. It is necessary that the star block copolymers have a minimum of 3 arms. In some embodiments, an increase in the number of arms can be advantageous, as more arms may help to provide a gel network with increased density or strength, or greater control over release of the bioactive agent encapsulated in the gelled composition. The higher the number of arms, the higher the molecular weight, however the smaller the overall size or diameter of the copolymer complex. Smaller micelles may help formation of a more dispersed micro structure in the gel providing a better dispersion of the drug, hence better encapsulation and better control over the release of the bioactive agent compared to structures formed with linear molecules.

In one form of the injectable composition, the balance between hydrophilic and hydrophobic blocks in the biodegradable, thermoreversible, non-linear block copolymer is such that the weight ratio between the hydrophilic and hydrophobic blocks is in the range of from about 10:1 to 1:10. In some embodiments, the weight ratio between the hydrophilic and hydrophobic blocks is in the range of from about 6:1 to 1:6, or from about 3:1 to about 1:3. The weight ratio of the hydrophilic and hydrophobic blocks may influence the physical properties of the injectable composition as well as its gelation behaviour.

The size or length of each hydrophobic or hydrophilic block may also influence the hydrodynamic diameter of the non-linear block copolymer. This in turn, can influence its viscosity and/or modulus. For instance, longer blocks and higher hydrodynamic diameters may give rise to higher viscosity. The balance between hydrophilicity and hydrophobicity may also play a role in the hydrodynamic diameter of the non-linear block copolymer. Without wishing to be limited by theory, it is believed that the presence of more hydrophilic blocks may lead to smaller diameters, and may lead to greater solubility being afforded to the bioactive agent included within the composition.

When the biodegradable, thermoreversible, non-linear block copolymer is a biodegradable star block copolymer of formula A(BC)ₙ, the weight ratio of B to C in the star block copolymer may be in the range of from 10:1 to 1:10. In some embodiments, the weight ratio of B to C may be from about 6:1 to about 1:6, or from 3:1 to about 1:3.

In some embodiments, one block present in each arm of the biodegradable star block copolymer may be a hydrophobic block while another block is a hydrophilic block. In biodegradable star block copolymers of formula A(BC)ₙ, B may represent a hydrophobic block and C represents a hydrophilic block. Alternatively B may represent a hydrophilic block and C represents a hydrophobic block.

Hydrophilic blocks present in the biodegradable, thermoreversible, non-linear block copolymer comprise a hydrophilic polymer. The hydrophilic polymer may have a molecular weight in a range selected from the group consisting of from about 100 to about 3000, from about 250 to about 2000, and from about 200 to about 1500.

Hydrophobic blocks present in the biodegradable, thermoreversible, non-linear block copolymer comprise a hydrophobic polymer. The hydrophobic polymer may have a molecular weight in a range selected from the group consisting of from about 500 to about 15,000, from about 1000 to about 10,000, and from about 1500 to about 5000.

In addition to gelling at physiological temperature, the non-linear block copolymer employed in the injectable composition is also biodegradable. This means that the non-linear block copolymer comprises at least one block that is biodegradable and comprises at least one biodegradable moiety. In some embodiments, at least one block of the non-linear block copolymer comprises a plurality of biodegradable moieties. One skilled in the art would understand that a biodegradable moiety is susceptible to degradation, cleavage or fragmentation under selected conditions, such as physiological conditions, resulting in the formation of lower molecular weight polymer fragments. Low molecular weight fragments may exhibit reduced cytotoxicity compared to high molecular polymers.

The non-linear block copolymer employed in the injectable composition of the invention is biocompatible as well as biodegradable.

In some embodiments, at least one of the hydrophilic and hydrophobic blocks present in the biodegradable, thermoreversible, non-linear block copolymer is biodegradable.

In some embodiments, the biodegradable, thermoreversible, non-linear block copolymer may comprise at least one non-biodegradable block in addition to the biodegradable block. For example, one of the hydrophilic and hydrophobic blocks may be biodegradable while the other of the hydrophilic and hydrophobic blocks is not biodegradable. In such embodiments, the non-biodegradable block remains biocompatible.

In one set of embodiments the hydrophobic block is biodegradable and thus comprises a biodegradable polymer. Biodegradable polymers comprise linkages that are susceptible to biodegradation, such as ester, amide and anhydride bonds. One skilled in the art would understand that such bonds may also be susceptible to degradation via hydrolysis under physiological conditions, producing lower molecular weight degradation products that can be readily metabolized by a subject and/or eliminated from the subject's body through normal excretory pathways. In such embodiments, the hydrophilic block may or may not comprise a biodegradable polymer.

In some embodiments, hydrophilic blocks present in the biodegradable, thermoreversible, non-linear block copolymer may comprise a hydrophilic polymer selected from the group consisting of poly(ethylene glycol), poly(ethylene oxide), poly(propylene oxide)poly(vinyl alcohol), poly(vinylpyrrolidone), poly(ethyloxazoline), polysaccharides or carbohydrates such as hyaluronic acid or dextran.

In some embodiments the hydrophilic block comprises a polyether. Exemplary polyethers may be derived from C2-C3 diols, and may be selected from the group consisting of poly(ethylene glycol), poly(propylene glycol), and copolymers thereof. In one set of the embodiments the hydrophilic block comprises poly(ethylene glycol) (PEG). A hydrophilic block comprising PEG has an advantage of being non-toxic and biocompatible and readily eliminated from a subject's body. The hydrophilic block may further be resorbable.

In biodegradable star block copolymers of formula A(BC)ₙ, C may be a hydrophilic block comprising a polyether as described herein.

In some embodiments, hydrophobic blocks present in the biodegradable, thermoreversible, non-linear block copolymer may comprise a polymer formed from at least one monomer selected from the group consisting of hydroxy acids (such as lactic acid or glycolic acid), cyclic monomers (such as caprolactone), amino acids, anhydrides, orthoesters, phosphazenes, phosphates, polyhydroxy acids, and mixtures thereof. The hydrophobic block may comprise a polymer that is biodegradable as well as being hydrophobic.

In some embodiments the hydrophobic block comprises a polyester. Polyesters are degradable in a physiological environment and can be tailored to provide controlled degradation by adjusting the composition and/or molecular weight of the polyester. In one set of embodiments the hydrophobic block comprises a polyester formed from at least one monomer selected from the group consisting of D,L-lactide, D-lactide, L-lactide, D,L-lactic acid, D-lactic acid, L-lactic acid, glycolide, glycolic acid, ε-caprolactone, ε-hydroxy hexonoic acid, γ-butyrolactone, γ-hydroxy butyric acid, δ-valerolactone, δ-hydroxy valeric acid, hydrooxybutyric acids, malic acid, mandelic acid and mixtures thereof. One skilled in the art would understand that polyesters formed from such monomers comprise a degradable ester moiety that is susceptible to degradation via hydrolysis. Hydrolysis may be mediated by a change of environmental conditions (e.g. a change in pH), or through the action of enzymes (enzyme-mediated hydrolysis).

In some embodiments the hydrophobic block comprises a polyester selected from the group consisting of poly(lactic acid), poly(glycolic acid), poly(caprolactone), and copolymers thereof. Examples of copolymers include poly(lactic acid-co-glycolic acid) and poly(lactic acid-co-caprolactone).

In one set of embodiments the hydrophobic block comprises a polyester formed from the polymerisation of D,L-lactic acid and ε-caprolactone. In such embodiments, the hydrophobic block comprises a polyester copolymer which is poly(lactic acid-co-caprolactone). In such embodiments, the mole ratio of lactic acid to caprolactone in the polyester copolymer may be in the range of from 90:10 to 10:90, preferably from 75:25 to 25:75.

In biodegradable star block copolymers of formula A(BC)ₙ, B may be a hydrophobic block comprising a polyester as described herein.

In one embodiment the biodegradable, thermoreversible, non-linear block copolymer is a biodegradable star block copolymer of formula A(BC)ₙ, where B comprises a polyester and C comprises a polyether. Exemplary polyesters and polyethers are described herein.

The blocks present in each arm of the biodegradable star block copolymer may be connected to one another via a linking group. The linking group may be a divalent group that covalently links one block with another block. When the biodegradable star block copolymer is of formula A(BC)ₙ, B and C may be connected via a linking group. The linking group present in the non-linear block copolymer may be derived from a suitable linking compound, such as a diisocyanate, for example, hexamethylene diisocyanate (HDI).

In order for different blocks (such as B and C) present in the arms of the star copolymer to be connected to one another via a linking group, the different blocks are required to comprise a terminal reactive functional group that is capable of reacting with a suitable linking compound that provides the linking group. Reaction of terminal functional groups (for example, hydroxyl groups) present on each of the different blocks with the linking compound results in covalent linkage of the blocks via a linking group derived from the linking compound. One skilled in the art would be able to select an appropriate linking compound, having regard to the nature of the terminal functional groups present in the different blocks as well as any desirable physical properties that may be possessed by the linking compound.

In some embodiments, the linking compound used to form the linking group may be a suitable polyfunctional compound having functional groups that are complementary to the terminal reactive functional groups present on different blocks used to form the arms of the star copolymer. Polyfunctional linking compounds comprise at least two reactive functional groups. In some embodiments the linking compound may comprise three, four or more reactive functional groups. In one set of embodiments, the linking compound is difunctional and comprises two reactive functional groups.

Being “complementary” means that a functional group on one molecule (e.g. a linking compound) is able to covalently react with the functional group present on another molecule (e.g. a polymer block), resulting in formation of a covalent bond between the two molecules.

Functional groups present on the polyfunctional linking compound may be independently selected at each occurrence.

In some embodiments, functional groups present on the linking compound may each be independently selected from the group consisting of hydroxy (OH), carboxylic acid (COOH), carboxylic acid ester (COOR), carboxylic acid halide (COX) amino (NR¹R²) and isocyanate (NCO), where R is C₁-C₄ alkyl, X is halo and may be selected from the group consisting of F, Cl, Br and I, and R¹ and R² are each independently selected from the group consisting of H and C₁-C₄ alkyl.

In some embodiments, the linking compound is homofunctional, where the functional groups of the linking compound are each of the same type (e.g. all isocyanate functional groups). In other embodiments, the linking compound may be heterofunctional and comprise a mixture of two or more different types of functional groups (e.g. a mixture of amino and hydroxy functional groups).

In one set of embodiments the linking group may be derived from a difunctional linking compound. For example, the linking group may be derived from a linking compound selected from the group consisting of a diol, a dicarboxylic acid, a dicarboxylic acid ester, a dicarboxylic acid halide, a diamine, a diamide, a dithiol and a diisocyanate.

In some embodiments, the linking group may be derived from a cyclic compound capable of ring-opening. For example, a divalent linking group may be derived from a cyclic anhydride or a cyclic imide, such as succinic anhydride or succinimide. In such embodiments, a functional group present on a block may covalently react with the cyclic compound, resulting in ring-opening of the cyclic compound and the generation of a functional group on the ring-opened compound. The functional group formed on the ring-opened compound is subsequently able to react with another block and in this manner, covalently link two adjacent blocks together.

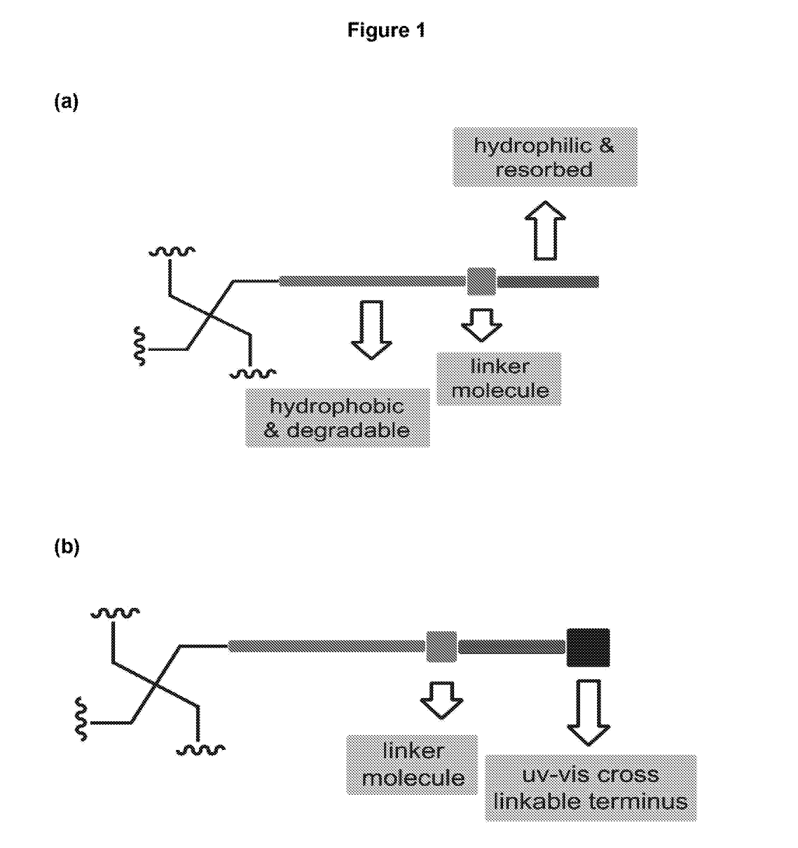

In particular embodiments when the biodegradable, thermoreversible, non-linear block copolymer is a biodegradable star block copolymer, a divalent linking group may covalently link the groups B and C in each arm of the star block copolymer (see FIG. 1b). The linking group may be derived from a difunctional linking compound or a cyclic compound as described herein. In one set of embodiments, the linking group may be derived from a diisocyanate. Examples of diisocyanates include hexamethylene diisocyanate (HDI), toluene diisocyanate (TDI), methylene bisphenyl diisocyanate (MDI) and ethyl lysine diisocyanate (ELDI).

In specific embodiments the linking group is derived from a diisocyanate linking compound, more specifically hexamethylene diisocyanate (HDI). A linking compound such as HDI is useful for providing a linking group as it is highly reactive at its terminal ends, requires less drastic reaction conditions and no reagents are required to enable reaction. The isocyanate is largely convenient to use due to its reactivity and affords cleaner products. The HDI backbone also consists of a linear hydrocarbon chain (hexane), which is hydrophobic by nature, and has a high degree of rotation due to six SP3 hybridised carbon atoms. When the non-linear block copolymer is a star block copolymer, the longer carbon-carbon chain afforded by the presence of the linking group and its higher degree of rotation can increase the flexibility of the arms of the star copolymer, which favours easy adoption of required confirmations during sol-gel transitions. Such properties built into the molecule in principle could enable rapid and unrestricted movement along the arms, leading to more defined and rapid phase changes from sol to gel.

Biodegradable star block copolymers comprising a linking group may be of formula A(BLC)ₙ, where A represents an n-valent core, one of B and C represents a hydrophilic block while the other of B and C represents a hydrophobic block, L represents a linking group, and n represents the numbers of arms extending from the core and is at least 3. Examples of hydrophilic blocks, hydrophobic block and linking groups (L) are described herein.

In biodegradable star block copolymers of formula A(BC)ₙ, the n-valent core (A) may be derived from a suitably functionalised multi-valent compound.

According to one set of embodiments, A is derived from pentaerythritol, or dimers or trimers of pentaerythritol. Examples of n-valent cores are shown below, where n is 4, 6 or 8:

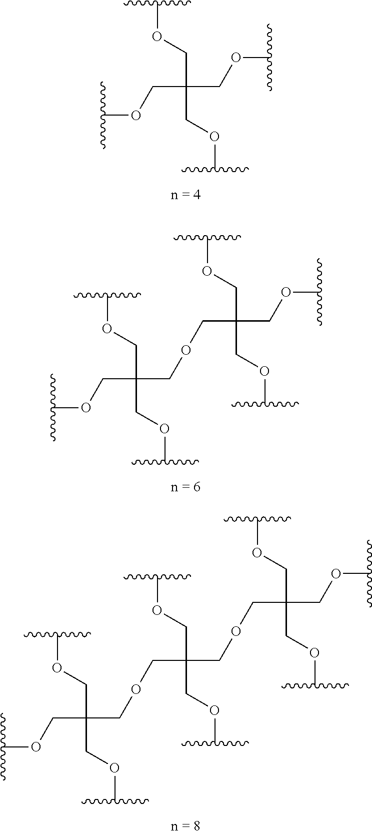 [SMILES: *OCC(CO*)(CO*)CO* ; *OCC(CO*)(CO*)COCC(CO*)(CO*)CO* ; *OCC(CO*)(CO*)COCC(CO*)(CO*)COCC(CO*)(CO*)CO*]

In one set of embodiments, biodegradable star block copolymers employed in the injectable compositions of the invention may have a structure of Formula (I), (II) or (III):

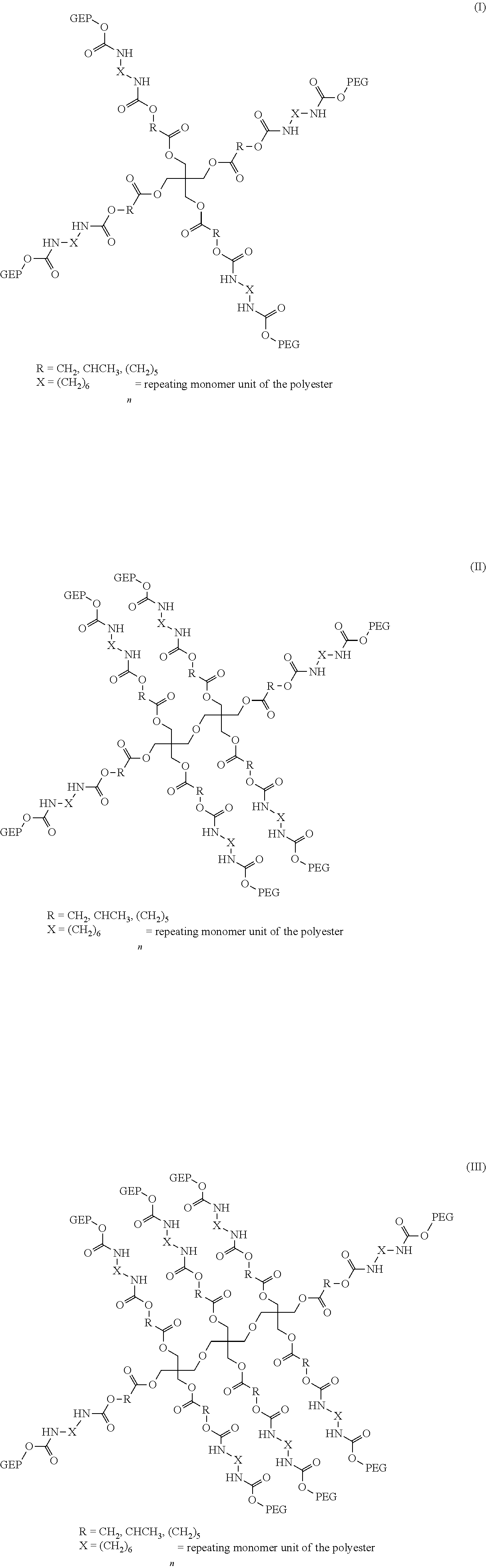

It can be desirable for each arm of the biodegradable star block copolymer to comprise the same type and arrangement of polymer blocks of similar molecular weight.

In some embodiments the biodegradable star block copolymer may comprise a further block (D) in each arm of the polymer. In such embodiments, the star block copolymer may be of formula A(BCD)ₙ. The composition of the further block (D) may be selected to provide additional functionality to the star block copolymer (e.g. introduction of crosslinkable groups), or provide further avenues to control the physical properties (e.g. gelation kinetics or compatibility) of the star block copolymer.

When the biodegradable, thermoreversible, non-linear block copolymer is a biodegradable star block copolymer, in some embodiments the star copolymer may be prepared by reacting a multi-functional core compound with a monomer composition comprising one or more monomers under conditions allowing polymerisation of the monomers and covalent attachment of a plurality of initial polymer blocks to the core. In some embodiments, the multi-functional core compound may initiate polymerisation of the monomers to result in covalent attachment of an initial polymer block to the core. The initial blocks each form part of an arm of the star block copolymer.

In one set of embodiments, the process may comprise the step of reacting an n-valent core with one or more monomers under conditions of condensation polymerization to provide an intermediate molecule comprising a plurality of initial blocks covalently attached to the n-valent core. The intermediate molecule may be of formula A(B)ₙ, where A represents an n-valent core, B represents an initial block and n represents the number of initial blocks attached to the core and is an integer of at least 3.

In another set of embodiments, a polymer block of desired composition may be pre-formed then subsequently reacted with a multi-functional core compound to result in covalent attachment of the pre-formed polymer block to the core compound. The polymer block attached to the core compound forms an initial polymer block. Preferably, a plurality of initial polymer blocks is attached to the core.

The initial blocks present may each comprise a terminal functional group. The terminal functional group may be capable of participating in covalent bonding reactions with a suitable linking compound to form a linking group precursor to the end of the initial block. According to one form, the terminal functional group may be selected from the group consisting of hydroxy (OH) and amino (NR¹R², where R¹ and R² are each independently selected from the group consisting of H and C₁-C₄ alkyl).

In exemplary embodiments, each initial block may comprise a polyester. The polyester may be formed from the polymerization of hydroxy acid monomers and/or cyclic monomers as described herein under condensation polymerization conditions. When an initial block comprises a polyester, a hydroxy group (OH) may be present as a terminal functional group of the initial block.

The initial blocks may then be coupled with a pre-formed block of desired composition, resulting in attachment of a subsequent block to the initial block and formation of a block copolymer in each arm of the biodegradable star copolymer. In embodiments where B represents an initial block, B may be coupled to a subsequent block represented by the group C, to form a block copolymer of formula (BC). The resulting star block copolymer is then of formula A(BC)ₙ, where each arm of the star copolymer comprises a block copolymer.

The pre-formed block desirably comprises a terminal functional group which is capable of participating in covalent bonding reactions with complementary functional groups to enable the subsequent block to be coupled with the initial block. In some embodiments the subsequent block comprises a polyether. The polyether may comprise a terminal hydroxy group. The hydroxy group is capable of reacting with complementary functional groups, such as carboxylic acid, carboxylic acid ester, carboxylic acid halide and isocyanate groups.

In one set of embodiments, the initial block may be directly coupled with the subsequent block. In such embodiments, the terminal functional group present on both the initial block may be complementary with the functional group present on the pre-formed block forming the subsequent block.

In another set of embodiments, the initial block and the subsequent block may be coupled via a linking group. The linking group may be derived form a suitable linking compound. Examples of linking compounds are described herein. In such embodiments, a terminal functional group present on the initial block reacts with the linking compound, resulting in covalent attachment of a linking group precursor at the end of the initial block. The resultant linking group comprises a functional group. The functional group of the linking group precursor is complementary to a functional group present on a pre-formed block selected to provide a subsequent block. Reaction of the functional group of the linking group precursor with the functional group of the pre-formed block results in attachment of a subsequent block. In this manner, the initial block and the subsequent block are coupled together via the linking group.

In one set of embodiments, the linking compound is a polyfunctional linking compound comprising a functional group complementary to functional group present on the initial block. In embodiments when the initial block comprises a hydroxy group as a terminal functional group, the polyfunctional compound may comprise a complementary functional group selected from the group consisting of carboxylic acid (COOH), carboxylic acid ester (COOR, where R is C₁-C₄ alkyl), carboxylic acid halide (COX, where X is halo such as F, Cl, Br and I) and isocyanate (NCO). In one embodiment, the polyfunctional linking compound is a diisocyanate. The isocyanate groups are capable of reacting with hydroxy groups present on an initial block and a subsequent block.

In another set of embodiments, the linking compound may be a cyclic compound that is capable of ring opening upon covalent reaction with the terminal functional group present on an initial block. Examples of cyclic compounds that may be used include cyclic anhydrides and cyclic imides, such as succinic anhydride or succinimide. The ring-opened compound forms a linking group at the terminal end of the initial block. For example, succinic anhydride is capable of undergoing ring-opening to provide an ethylene (C₂) linking group having a terminal carboxylic acid functional group. The terminal carboxylic acid group is capable of undergoing a covalent reaction with a complementary functional group on a pre-formed block to thereby attach a subsequent block.

One approach for synthesizing biodegradable star block copolymers is shown in Scheme 2 below.

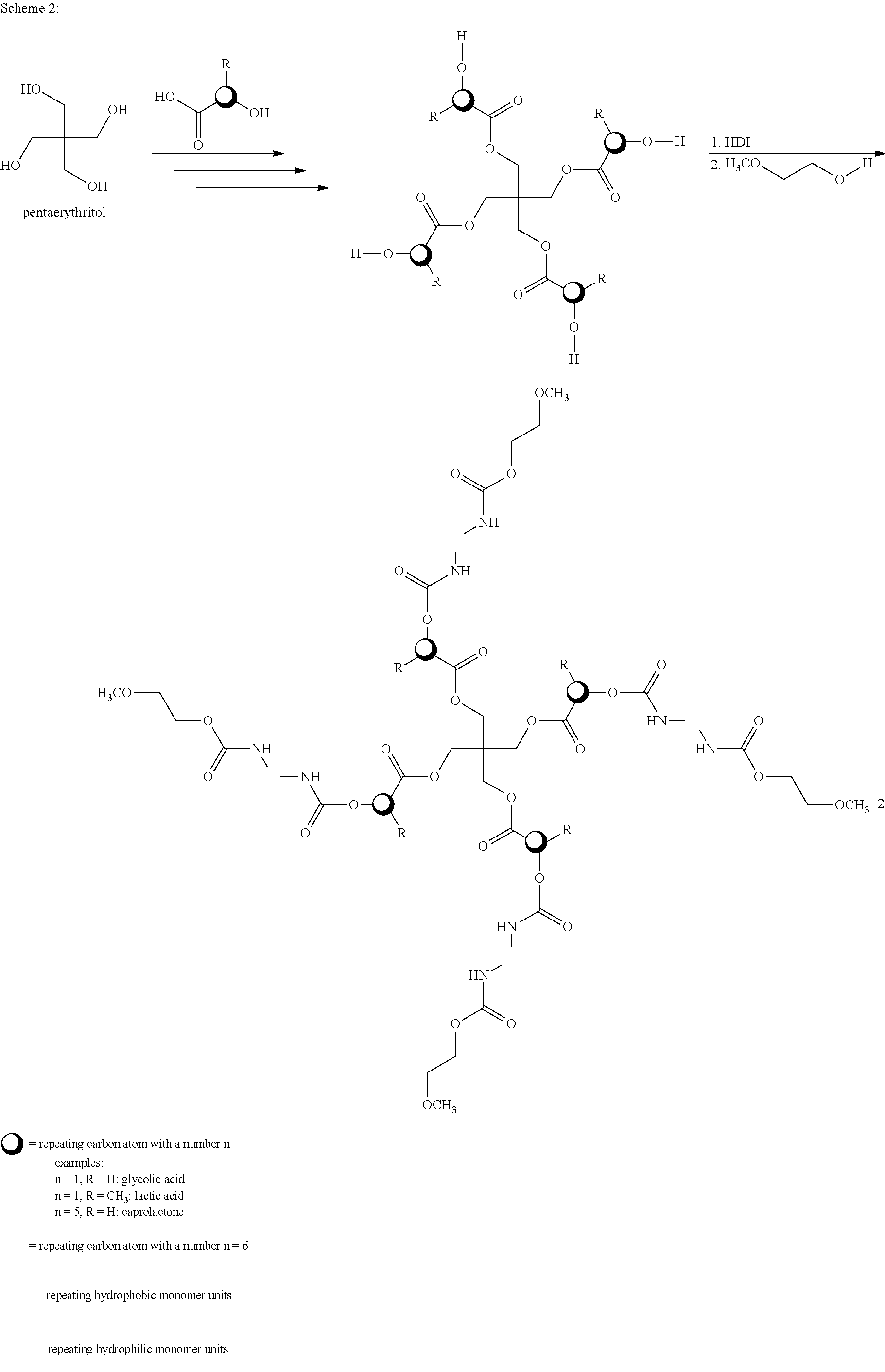

As illustrated above, the multi-functional core compound pentaerythritol (a polyol) can be reacted with a monomer composition comprising a mixture of lactic acid and caprolactone (hydroxy acid monomers) to covalently bond 4 initial blocks comprising poly(lactic acid-co-caprolactone) to the pentaerythritol core. A diisocyanate linking compound, hexamethylene diisocyanate (HDI), is then reacted with the poly(lactic acid-co-caprolactone) blocks to provide a linking group having an isocyanate functional group at the terminal end of each poly(lactic acid-co-caprolactone) block. The isocyanate group of the linking group is then covalently reacted with pre-formed blocks comprising poly(ethylene glycol)methyl ether to covalently bond a subsequent block comprising poly(ethylene glycol) to each initial poly(lactic acid-co-caprolactone) block.

In some embodiments the biodegradable star block copolymer may comprise a further block (D) and be of formula A(BCD)ₙ. When desired, the further block (D) may be introduced into each arm of the star block copolymer by covalently reacting a terminal functional group present on the block C with a complementary terminal functional group on the further block (D), in order to attach C to D. In other embodiments, the block C may be modified to provide a linking group at the end of the block C prior to coupling with D. The linking group may comprise a terminal functional group, which can react with a complementary functional group present in the further block (D), resulting in covalent coupling of D to C via the linking group.

In some embodiments, the biodegradable star block copolymers may further comprise crosslinkable groups at the terminus of one or more arms. The crosslinkable groups may permit molecules of star block copolymer to be covalently crosslinked after administration of the composition, leading to compositions that once administered to a subject, cannot revert back to a liquid state. Thus whilst they are thermoreversible compositions for the purpose of being injectable and become gels after administration, once gelled and crosslinked, they no longer display “reversible” behaviour. Chemically or ionically crosslinkable groups known in the art may be used. In some embodiments the crosslinkable groups are polymerisable under free radical conditions. In such embodiments polymerisation of the crosslinkable groups may occur following photoinitiation with visible or ultraviolet radiation, or after thermal initiation by the application of heat. This activation and polymerisation of the crosslinkable groups occurs after administration of the composition. Examples of crosslinkable groups that may be used include unsaturated groups such as vinyl groups, allyl groups, cinnamates, acrylates, diacrylates, oligoacrylates, methacrylates, dimethacrylates, oligomethacrylates, or other biologically acceptable polymerizable groups. The ability to crosslink the gelled composition after it has been formed can facilitate formation of a strong gelled composition that is complimentary in size and shape to the administration area or defect into which the composition is injected.

Biodegradable, thermoreversible, non-linear block copolymers having other architectures, such as branched, hyperbranched, comb, brush and dendritic architectures, may also be employed in the injectable composition of the invention. Non-linear block copolymers of such architectures may be prepared by a skilled person using conventional techniques known in the relevant art. Some examples of synthetic protocols that may be employed are described in Reactive and Functional Polymers, 71, 245-253, 2011.

In embodiments of the invention the injectable composition comprises from about 3% to about 50% by weight of the biodegradable, thermoreversible, non-linear block copolymer. In some embodiments the injectable composition may comprise from about 5% to about 40% or from about 10% to about 30% by weight of the non-linear biodegradable block copolymer. In order to obtain a viable gel phase transition with the copolymer, a certain minimum concentration, e.g. 3% by weight, is required. In some embodiments a higher polymer concentration may be preferred as a stronger or more stable gel network may be formed.

One advantage associated with the use of a non-linear block copolymer, such as a star block copolymer, is that it is possible to form a composition that comprises a high polymer content yet is still flowable and injectable. In the case of biodegradable star block copolymers, the multi-arm polymer architecture enables a polymer solution having a lower viscosity than that of a polymer solution comprising a corresponding linear polymer of similar molecular weight, to be prepared. The lower viscosity can improve the injectability of the compositions through narrow restrictions, such as narrow gauge needles. In one set of embodiments it is desirable for the composition of the invention to be injectable through a 28 gauge needle. In one set of embodiments, the injectable composition of the invention has a viscosity in the range of from about 0.001 to 2 Pa*s when in liquid form, with a corresponding storage modulus reflecting its gel-like properties.

One other advantage associated with the use of non-linear block copolymer, such as a star block copolymer, is that a more stable gel structure can be achieved due to the ability to incorporate higher polymer solids content in the injectable composition. For instance, literature reports suggest that regular micellar structures might be more readily formed with star polymers, compared with linear polymer of similar molecular weight (J. Polym. Sci. Part A, 2006, 44, pages 888-899). Without wishing to be limited by theory, it is believed that more uniform micelle structures that can be provided by star polymers can assist in the formation of a gel micro-structure that consequently aids the dispersion and encapsulation of a bioactive agent in the composition of the invention, and allow improved control of release of the bioactive agent to be achieved.

The injectable composition of the invention also comprises an aqueous solvent. The aqueous solvent may be water, or water in admixture with a pharmaceutically acceptable water-soluble solvent. An example of a pharmaceutically acceptable water-soluble solvent is ethanol.

The injectable composition may comprise from about 50% to about 97% (w/w) of aqueous solvent. In one set of embodiments, the injectable composition comprises a high proportion of aqueous solvent, for example, at least 60%, or at least about 70%, by weight of solvent.

Where the injectable composition comprises water, the resulting gelled composition may in some embodiments be considered to be a hydrogel.

It is preferable that the biodegradable, thermoreversible, non-linear block copolymer be soluble in the aqueous solvent such that the resulting injectable composition is homogeneous, with little or no phase separation of the copolymer evident.

The injectable composition of the invention also comprises a bioactive agent. The bioactive agent may be selected from a range of bioactive agents or medically useful drugs or vaccines of all types for use in the treatment or prophylaxis of diseases or disorders, and the present invention is not limited to specific bioactive agents. Examples of bioactive agents that may be incorporated in and delivered by the injectable composition of the invention are described in references as the Merck Index, the Physicians Desk Reference, and The Pharmacological Basis of Therapeutics. They include hydrophilic drugs, hydrophobic drugs, proteins or antibodies, hormones, genes, or nucleic acids, oligonucleotides, active agents for antisense therapy, polysaccharides and other sugars, lipids, gangliosides, vasoactive agents, neuroactive agents, anticoagulents, immunomodulating agents, anti-cancer agents, anti-inflammatory agents, antibiotics, antivirals and vaccines.

In some embodiments, the bioactive agent may be selected from the group consisting of anti-cancer agents such as actinomycin D, anastrozole, azacitidine, bevacizumab, bicalutamide, bleomycin, BCNU, bortezomib, camptothecin, capecitabine, carboplatin, cetuximab, daunorubicin, dasatinib, docetaxel, doxorubicin, epirubicin, erlotinib, exemestane, gefitinib, gemcitabine, goserelin, imatinib, STI-571, irinotecan, lapatinib, letrozole, leuprolide, methotrexate, mitomycin, oxaliplatin, paclitaxel, pemetrexed, rituximab, sorafenib, sunitinib, tamoxifen, taxotere, tegafur-uracil, temozolomide, trastuzumab, triptorelin, vinorelbine, porcabazine, dacarbazine, altretamine, displatin, mercaptopurine, thioguanine, fludarabine phosphate, cladribine, pentostatin, fluorouracil, cytarabine, azacitidine, vinblastine, vincristine, etoposide, teniposide, topotecan, dactinomycin, idarubincin, plicamycin, flutamide, leuprolide, gasoerelin, aminoglutethimide, amsacrine, hydroxyurea, asparaginase, mitoxantrone, mitotane, retinoic acid derivative, bone marrow growth factors amifostine, carmustine, lomustine, semustine, anti-VEGF agents and the like; antipsychotics such as olanzapine and ziprasidone; antibacterials such as cefoxitin; anthelmintics such as ivermectin; antivirals such as acyclovir, immunosuppressants such as cyclosporin A (cyclic polypeptide-type agent), steroids, and prostaglandins; cardiovascular drugs such as dipyridamole, and eptifibatide.

In some embodiments, the bioactive agent may be a small molecule. Such bioactive agents may have a molecular mass of no more than 2000 Da, no more than about 1500 Da, or no more than about 1000 Da.

In some embodiments, the bioactive agent may be a large molecule. Such bioactive agents may have a molecular mass of more than 2 kDa, more than 5 kDa, more than 10 kDa, more than 50 KDa, or more than 100 kDa.

Polypeptide and protein drugs may also be particularly suitable for inclusion in the injectable composition for delivery to a subject. Examples of pharmaceutically useful polypeptides and proteins may be erythropoietin, follistatin, oxytocin, vasopressin, adrenocorticotropic hormone, epidermal growth factor, platelet-derived growth factor (PDGF), prolactin, luliberin, luteinizing hormone releasing hormone (LHRH), LHRH agonists, LHRH antagonists, growth hormone (human, porcine, bovine, etc.), growth hormone releasing factor, insulin, somatostatin, glucagon, interleukin-2 (IL-2), interferon-α, β, or γ, gastrin, tetragastrin, pentagastrin, urogastrone, secretin, calcitonin, enkephalins, endorphins, angiotensins, thyrotropin releasing hormone (TRH), tumor necrosis factor (TNF), nerve growth factor (NGF), granulocyte-colony stimulating factor (G-CSF), granulocyte macrophage-colony stimulating factor (GM-CSF), macrophage-colony stimulating factor (M-CSF), heparinase, bone morphogenic protein (BMP), hANP, glucagon-like peptide (GLP-1), interleukin-11 (IL-11), renin, bradykinin, bacitracins, polymyxins, colistins, tyrocidine, gramicidins, cyclosporins or synthetic analogues, modifications and pharmacologically active fragments thereof, enzymes, cytokines, antibodies or vaccines.

The injectable composition of the invention comprises a suitable amount of bioactive agent. In some embodiments the injectable composition comprises from about 0.01% to about 20% by weight of bioactive agent. However, one skilled in the art would understand that the amount incorporated in the injectable composition will depend on the nature of the bioactive agent. The injectable composition may contain a lower concentration of bioactive agent compared to drug delivery compositions of the prior art.

One benefit of the injectable compositions of the invention is the ability of the non-linear block copolymer to increase the solubility of many bioactive agents. Biodegradable star block copolymers employed in injectable compositions of the invention have a combination of hydrophobic block(s) and hydrophilic block(s), which provide distinct hydrophilic and hydrophobic domains and renders the block copolymer amphiphilic. The presence of hydrophilic and hydrophobic domains can assist in stabilizing and solubilizing hydrophilic or hydrophobic bioactive agents, respectively, in the composition. For hydrophobic bioactive agents such as paclitaxel, the ability to increase the solubility of the drug (and hence its ultimate bioavailability) can aid in its delivery to a patient.

In some embodiments, the injectable composition of the invention may further comprise one or more excipients (buffer salts for example), additives and/or adjuvants. The excipient, additive or adjuvant generally would not contribute to the formation of the gel network, but may associate with either a hydrophilic or hydrophobic domain of the biodegradable, thermoreversible, non-linear block copolymer and thus enhance the properties of that domain and affect the properties of the composition, such as viscosity, injectability, consistency or storage modulus. Excipients, additives or adjuvants present in the injectable composition may also assist to enhance the solubility of the bioactive agent.

In some embodiments the injectable composition may comprise an additive selected from the group consisting of a polysaccharide, a polyether, and mixtures thereof. Polysaccharides and polyethers are generally non-toxic and hydrophilic and would associate with a hydrophilic domain of a biodegradable, thermoreversible, non-linear block copolymer. Partitioning of an additive, such as a polysaccharide and/or polyether, in the hydrophilic domain may assist to help further control release of bioactive agents. In this regard, the polysaccharide and/or polyether may be considered to be a release control additive that facilitates the ability to control the release of the bioactive agent from the gelled composition of the invention. The inclusion of an additive in the injectable composition may also help to improve the consistency of the gelled composition in some embodiments.

Exemplary polysaccharides include but are not limited to xanthan gum, welan gum, dextran, gellan, pullulan, guar gum, locust bean gum, chitin and alginate.

An exemplary polyether is poly(ethylene glycol) (PEG). The poly(ethylene glycol) may have a molecular weight in the range of from 200 to 3000. In some embodiments, the polyether is poly(ethylene glycol) (PEG) having a molecular weight selected from the group consisting of 350, 550, 750, 1000 and 2000.

In preparing the injectable composition, the biodegradable, thermoreversible, non-linear block copolymer is dispersed in the aqueous solvent to form a solution comprising the block copolymer. The bioactive agent may then be added and mixed into the polymer containing solution. When an excipient, additive and/or adjuvant is employed in the composition, the excipient, additive or adjuvant or combinations thereof may be added to the solution before or after the bioactive agent is mixed into the solution.

In some embodiments, the bioactive agent may undergo a treatment step that conditions the bioactive agent prior to being mixed with the biodegradable, thermoreversible, non-linear block copolymer. Treatment of the bioactive agent may help to enhance its interaction with the copolymer. In one form, the bioactive agent is firstly treated by sonication, then heating and then cooling. Improved interactions with the copolymer, for example more uniform dispersion or greater entanglement of the bioactive agent in the copolymer matrix, may aid in the sustained delivery of the bioactive agent. Treatment of the bioactive agent may be particularly beneficial when the bioactive agent is a pharmaceutically useful protein or peptide having a molecular weight of more than 10 kDa.

In some embodiments, the injectable composition comprising the bioactive agent may undergo a treatment step that enhances mixing and interaction of the bioactive agent with the biodegradable, thermoreversible, non-linear block copolymer. In one form, the injectable composition is treated by sonication, then heating and then cooling to facilitate interactions of the bioactive agent with the non-linear block copolymer.

The injectable composition of some embodiments is a liquid at room temperature (approximately 20° C.) and can be injected at room temperature to administer the composition to a subject. Injection can be possible due to the biodegradable, thermoreversible, non-linear block copolymer having a low viscosity.

In another set of embodiments, the injectable composition may be a viscous liquid under static conditions at room temperature (approximately 20° C.). As discussed above, such an injectable composition may be thixotropic, such that the composition is able to be converted to a flowable liquid when the composition is subjected to shear, such as when the composition is injected through the lumen of a needle or a catheter.

Administration of the injectable composition can be achieved using a range of surgical techniques or devices. It is preferred that the surgical technique or device be minimally invasive. For example, the injectable composition may be administered to a desired site via a catheter delivery system or via a small gauge needle with an internal diameter of approximately 0.2 mm (28 gauge) or less. The ability to administer the injectable composition by minimally invasive techniques significantly reduces the mechanical irritation or trauma that may be suffered by a subject, and can help with patient compliance and ease of delivery.

Once administered to a desired site in a subject, the injectable composition spontaneously gels at physiological temperature to form a gelled composition in situ. The gelled composition encapsulates the bioactive agent contained in the injectable composition. Spontaneous gelation of the injectable composition occurs as a result of the biodegradable, thermoreversible, non-linear block copolymer present in the injectable composition self-assembling into micelles that subsequently aggregate to form a gel network. A preferred block copolymer is a biodegradable star block copolymer as described herein. The gelled composition can form an implant at the site of administration.

In one set of embodiments, the injectable composition of the invention has a storage modulus in the range of from about 1 to 20 Pa when in liquid form and when in gel form at physiological temperature. The injectable composition of the invention can display variable storage modulus as the composition changes between liquid and gel form. In some embodiments, the injectable composition has a storage modulus of from about 1 to 2 Pa when in liquid form. During the transition of the liquid composition to a gel state with increasing temperature, the storage modulus increases until the gelled composition is formed at physiological temperature. The gelled composition has a storage modulus and viscosity that is higher than that of the liquid composition. In one set of embodiments, the gelled composition of the invention has a storage modulus in a range selected from the group consisting of from about 8 to 20 Pa, about 10 to 15 Pa and about 12 Pa. As the composition of the invention is thermoreversible, the storage modulus of the composition can return to being in the range of from about 1 to 2 Pa when the gelled composition reverts to liquid form.

Once the injectable composition has gelled, the gelled composition forms a depot from which an effective amount of the bioactive agent can be released. In one set of embodiments an effective amount of the bioactive agent is released from the gelled composition over a period of at least 7 days, at least 14 days, at least 21 days, at least 28 days, at least 35 days, at least 45 days, at least 60 days or at least 90 days. Thus, the bioactive agent is able to exert its desired biological or pharmacological effect over a sustained period of time.

The injectable composition and the gelled composition formed therefrom can provide a desired rate (or phases) of release of the bioactive agent. In some embodiments the gelled composition can provide for the release of the bioactive agent to occur in a single phase, or in multiple phases.

In some embodiments, release of the bioactive agent from the gelled composition may be substantially zero order. In some embodiments the gelled composition can provide for release of the bioactive agent with little or no initial burst effect. No burst effect may be defined as being less than 30% of the encapsulated bioactive agent being released within the first 24 hours after gel formation. In some embodiments, the gelled composition provides release of no more than about 20%, preferably no more than about 10%, and most preferably no more than about 5% of the bioactive agent, within the first 24 hours after gel formation.

In some embodiments, release of the bioactive agent from the gelled composition occurs in at least two separate and distinct rates or phases and in some embodiments, may occur in three separate rates or phases. A release phase may be characterised by a defined release mechanism or release kinetics. A release phase may be related to the quantity of bioactive agent released in that particular phase. In one set of embodiments, release of the bioactive agent within a phase may be substantially zero order.

In some embodiments, release of the bioactive agent from the gelled composition may occur in two phases (i.e. biphasic), and may be initially burst release followed by diffusion controlled release. In some alternative embodiments, release of the bioactive agent from the gelled composition may occur in three phases (i.e. triphasic) and may be initially burst release followed by diffusion controlled release and finally, release under degradation control.

The rate and period of bioactive agent release may be influenced by the composition and/or properties of the biodegradable, thermoreversible, non-linear block copolymer, as well as the concentration of polymer in the injectable composition. The bioactive agent release profile may also be influenced by the presence of an additive or adjuvant in the injectable composition. Accordingly, these parameters may be adjusted to achieve a desired release profile for a selected bioactive agent to aid in the treatment or prophylaxis of a disorder or disease.

Without wishing to be limited by theory, it is believed that the use of a biodegradable, thermoreversible, non-linear block copolymer having hydrophilic and hydrophobic domains in the injectable composition aids in the controlled release of hydrophilic and hydrophobic bioactive agents. For example, it is believed that hydrophobic bioactive agents will be attracted to hydrophobic domains in the non-linear block copolymer and hence will be retained within the gelled polymer structure in preference to partitioning into a surrounding aqueous environment. This in turn, is believed to contribute to slower and more sustained release of the bioactive agent from the gelled composition over a longer period of time. In this manner, the composition of the non-linear block copolymer may therefore influence the rate of release of the bioactive agent from the gelled composition via diffusion mechanisms.

For hydrophilic bioactive agents, controlled and sustained release may be achieved by making the environment of the gelled polymer structure more hydrophilic and hence more attractive to the bioactive agent. In some embodiments, this may be achieved by incorporating a hydrophilic additive such as xanthan gum into the injectable composition of the invention. The hydrophilic additive may associate with hydrophilic domains in the non-linear block copolymer and hence help to attract hydrophilic bioactive agents to those domains in preference to the bioactive agent partitioning into the surrounding environment.

Release of the bioactive agent from the gelled composition may initially be burst release followed by release under diffusion control. Diffusion controlled release can be influenced by the relative affinity of the bioactive agent for the biodegradable, thermoreversible, non-linear block copolymer compared to the surrounding environment. After a period of time following administration and gel formation, release of the bioactive agent may also be influenced by the rate of biodegradation of the gelled composition. In some embodiments, release may be dictated by a combination of diffusion and degradation control if biodegradation of the gelled composition commences while release of the bioactive agent is predominately under diffusion control. Bioactive agent release via biodegradation of the gelled composition in a physiological environment can represent the final phase of delivery. It may be possible to control this final phase by appropriate selection of the composition of the biodegradable blocks present in the non-linear block copolymer.

In addition to bioactive agent release, other properties such as gel strength, gelation temperature and degradation rate can also be controlled through design and preparation of the various copolymer blocks, namely, through modifications of the weight percent of hydrophilic blocks and hydrophobic blocks, the mole percentages of the monomer units (e.g. caprolactone, glycolic acid, lactic acid) present in each type of block, and the molecular weight and polydispersity of the biodegradable, thermoreversible, non-linear block copolymer.

In use, the injectable composition of the invention can be administered in the vicinity of a site requiring treatment to provide for localized delivery of a bioactive agent at the desired site, rather than intravenously. Such localized delivery may be advantageous when the bioactive agent is a highly potent bioactive agent, such as for example a cancer drug, as it reduces the possibility of organ damage or other serious side effects that may result from non-selective treatment arising from intravenous administration of the drug. In other embodiments, the injectable composition may be used for systemic delivery of a bioactive agent.

One benefit of the injectable composition of the invention is that it can provide for release of an effective amount of a bioactive agent over a longer period of time than compositions of the prior art. Sustained release of the bioactive agent means that the time for bioactive efficacy can be increased, while the dose of drug required to be administered may be reduced due to enhanced delivery.

The ability to achieve sustained release of a bioactive agent may also reduce the need for repeated administration of the bioactive agent, thus reducing the potential for any trauma to a subject that may arise from repeated administrations. This may be advantageous in instances where bioactive agents need to be delivered frequently (as in the case of protein drugs) due to rapid degradation and clearance of the bioactive agent from the site of treatment.

One other advantage of the invention is that the gel network formed with the injectable composition of the invention is soft, pliable and deformable due to the high aqueous solvent content of the injectable composition, which is retained in the resulting gel. Thus discomfort associated with solids injection or solid implants can also be reduced.

As the gelled composition is formed with a biodegradable copolymer, the composition is capable of degrading to non-toxic metabolites in vivo, such that removal of the gel from a subject's body is not necessary once the bioactive efficacy is exhausted. Rather, the degradation products can be eliminated from the body through normal excretory pathways. For example, polyester blocks in the non-linear block copolymer can be biodegraded to caproic, lactic acid, glycolic acid, and other corresponding metabolites within a specific time interval. Furthermore, the polyethylene glycol blocks can be removed from the body of a subject by excretion.

The use of a biodegradable, thermoreversible, non-linear block copolymer also provides a further avenue for controlling delivery of a bioactive agent encapsulated in the gelled composition as a result of degradation or erosion of the polymer in the physiological environment over time, which allows further amounts of the bioactive agent to be released.

In another aspect, the present invention provides a method of treating or preventing a disease or disorder in a subject, the method comprising administering an injectable composition according to any one of the embodiments described herein to a subject. In one set of embodiments the method comprises injecting the composition from the lumen of a syringe to administer the composition to the subject.

In another aspect, the present invention also provides use of an injectable composition in accordance with any one the embodiments described herein in manufacture of a medicament for treatment or prophylaxis of a disease or disorder of a subject. In one set of embodiments the medicament is in the form of an implant.

In one set of embodiments the disease or disorder is cancer and the bioactive agent is an anti-cancer agent Examples of anti-cancer agents that may be delivered by the injectable composition are described herein.

In another aspect, the present invention a method of treating cancer in a subject comprising administering an injectable composition to the subject by injection, wherein the injectable composition comprises:
a biodegradable, thermoreversible, non-linear block copolymer;
an aqueous solvent; and
an anti-cancer agent,

wherein the composition is injectable as a liquid, whereby the liquid is converted to a gel at physiological temperature and wherein the gelled composition provides release of an effective amount of the anti-cancer agent over a period of at least 7 days.

The present invention also provides a method of treating or preventing an ocular disease or disorder in a subject. In one form, the ocular disease or disorder is age-related macular degeneration (AMD) and the method comprises administering an injectable composition as described herein to the subject by injection, wherein the injectable composition comprises at least one bioactive agent selected from the group consisting of bevacizumab, ranibizumab and pegaptanib sodium. Preferably the composition is administered directly to the vitreous humor of the eye by intravitreal injection.

The present invention also provides a method for preventing keloidal scars in a subject, the method comprising administering an injectable composition as described herein to the subject, wherein the injectable composition comprises follistatin as a bioactive agent. Preferably the injectable composition is topically applied.

### EXAMPLES

The present invention is described with reference to the following examples. It is to be understood that the examples are illustrative of and not limiting to the invention described herein.

### Materials

Pentaerythritol (PE) FW 136 (98%), Dipentaerythritol FW 254 (98%), Tripentaerythritol FW 372 (98%), Dibutyltin dilaurate (DBTL) FW 631.56 (98%) were purchased from Aldrich and used as received. Poly(ethylene)glycol methyl ether (PEG-O—CH₃) average MW 350, 550 were received from Aldrich and dried under 40° C. in vacuo (0.1 mmHg) overnight prior to use. ε-Caprolactone FW 114.14 (>99%) and DL-Lactic acid FW 90.08 (90% in water) were purchase from Fluka and used as received. Hexamethylene Diisocyanate (HDI) FW 168.2 (>98%) was received from Fluka and purified further by distillation under reduced pressure prior to use. p-Toluene sulphonic acid monohydrate (99%) was purchased from Acros Organics and used as received.

### Method

### A. Typical Synthesis of a Four Arm Polvester Olvol

### PE-LA-CL (75:25) 2100 MW

Pentaerythritol (PE) (3.2131 g, 1 mole), DL-Lactic acid (42.5177 g, 18 moles) (LA) and ε-Caprolactone (16.1622 g, 6 moles) (LC) was heated in a round bottom flask 160-170° C. in the presence of THF, 250 mL and 1.0 g of p-toluenesulphonic acid monohydrate. Reaction mixture at reflux was allowed to stir for 3 days at reflux and ambient pressure. The water generated was collected using a Dean-Stark apparatus. The solvent was decanted and the reaction mixture concentrated using a rotary evaporator and the residual solvent removed further under high vacuum to produce slightly yellow clear transparent product (˜80% yield) (Theoretical MW—2118.43, GPC MW as observed: Mn 2766, Mw 3469, Mp 3356, Mz 4266, PD 1.25). The below Scheme 3 shows the reaction used to prepare a core with an initial polymer block attached, with the repeating unit of the polymer block shaded. The characteristic of the polyester polyol is listed in Table 1.

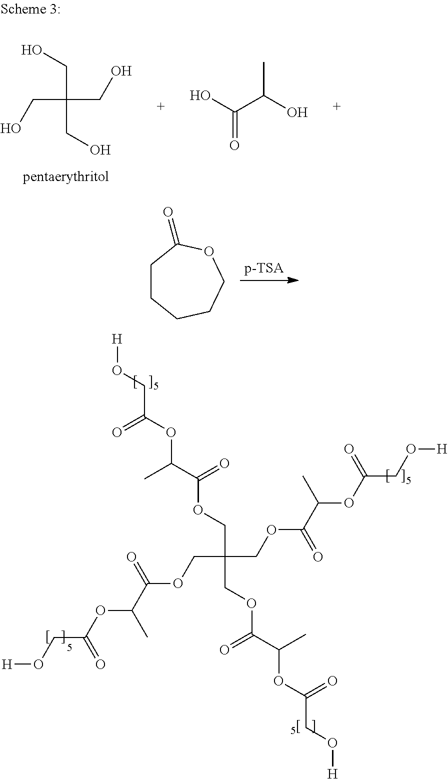

All other polyester polyols were synthesized following this procedure at the correct stoichiometric ratios. The core of the polyester polyols determined the number of arms of the polyester, pentaerythritol, dipentaerytritol and tripentaerythritol giving polymers with 4, 6 and 8 arms respectively. The molecular weight characteristics of the prepared polymers are shown in Table 1.

TABLE 1
Examples of typical polyester polyols prepared and their
characteristics

| Sample | Target | GPC | | |
|---|---|---|---|---|
| composition | Mn | Mn | Mw | PDI |
| P(LA₇₅CL₂₅)₄ | 2118 | 2766 | 3469 | 1.25 |
| | | 2818 | 3519 | 1.24 |
| | | 2785 | 3511 | 1.26 |
| P(LA₇₅CL₂₅)₄ | 4100 | 5198 | 6656 | 1.28 |
| | | 5413 | 6875 | 1.26 |
| P(LA₇₅CL₂₅)₄ | 10047 | 9990 | 13361 | 1.33 |
| | | 12456 | 15878 | 1.27 |
| P(LA₇₅CL₂₅)₄ (large scale) | 1999 | 2106 | 2762 | 1.31 |
| | 1999 | 2143 | 2686 | 1.25 |
| P(LA₇₅CL₂₅)₄ | 1999 | 2527 | 3156 | 1.25 |
| | 1999 | 2486 | 3067 | 1.23 |
| P(LA₇₅CL₂₅)₄ | 3109 | 3991 | 5063 | 1.27 |
| P(LA₇₅CL₂₅)₄ | 3888 | 4674 | 5608 | 1.20 |
| P(LA₁₀₀)₄ | 1999 | 2587 | 3171 | 1.22 |
| P(LA₇₅CL₂₅)₄ | 1999 | 2590 | 3173 | 1.22 |
| P(LA₇₅CL₂₅)₆ | 3227 | 3907 | 4637 | 1.19 |
| | 3227 | 3871 | 4606 | 1.18 |
| P(LA₁₀₀)₆ | 1550 | 1586 | 1799 | 1.13 |
| P(LA₁₀₀)₈ | 2100 | 2154 | 2401 | 1.11 |

B. Typical Functionalisation with Hexamethylene Diisocyanate (HDI)

The polyester polyol PE-LA-CL (75:25) (0.5×10³ moles) was dissolved in dry DCM 15 mL in a round bottom glass equipped with a magnetic stirrer bar and HDI (20×10³ moles, 10 fold excess) added at room temperature. The reaction mixture was stirred for 4 hrs and 10 mg of DBTL added. The mixture was allowed to stir at ambient temperature over night. The product was precipitated into dry n-heptane (1500 mL), decanted and the polymer residue immediately re-dissolved in DCM for the next functionalisation step. Functionalisation of the initial polymer block with HDI is shown in Scheme 4.

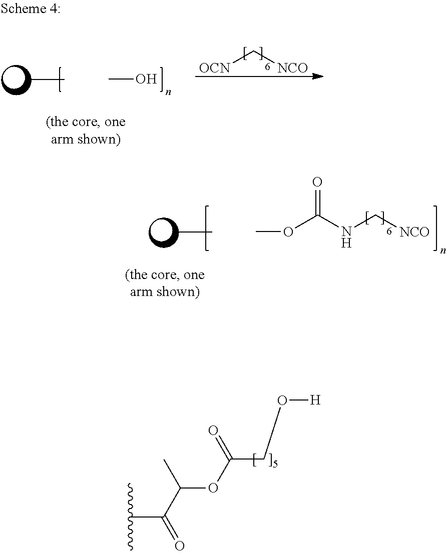

### C. Typical Procedure to Add the Second Block: PEG-OCH₃ 350 MW

The HDI functionalized polyester polyol is dissolved in dry DCM (15 mL) and pre-dried monomethyl PEG-O—H (3×10⁻³ moles, 1.5 equivalents) added at room temperature. The reaction mixture was stirred for 4 hrs followed by the addition of the catalyst DBTL (10 mg). The solution was allowed to stir over night at room temperature. The polymer product was precipitated into n-Heptane (1000 mL), the solvent decanted, the precipitated polymer re-dissolved in a minimum of DCM, transferred to a round bottom flash and the solvent removed by using a rotary evaporator. The residual solvents in the product polymers were removed by high vacuum to obtain the crude final polymer. Attachment of a subsequent block is shown in Scheme 5.

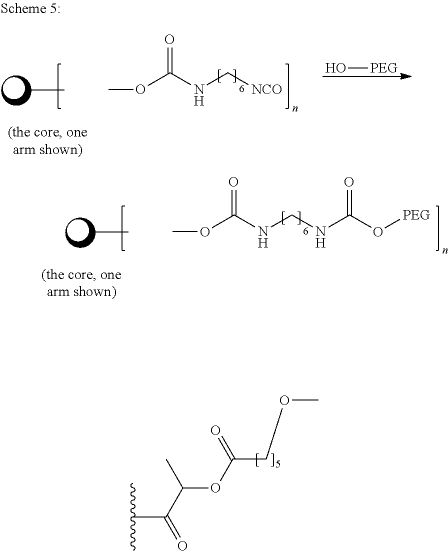

### D. Typical Purification Procedure:

The crude product polymers were dissolved in de-ionized water below 10° C. Upon complete dissolution the mixture was heated to 60° C. The polymers in the solution with the increase of the temperature precipitated and isolated. Three precipitations were carried out to afford the clean final polymer product.

Using the above methods, a range of biodegradable star block copolymers were prepared, as detailed in Table 2.

TABLE 2
Examples of functionalized TRPs prepared according to B-D
methodology and their characteristics

| | | | | | | PEG |
|---|---|---|---|---|---|---|
| | No. | Sample | GPC | | | content |
| Ex. | arms | composition | Mn | Mw | PDI | (th wt %) |
| 1 | 4 | P(LA₇₅CL₂₅(500)-HDI-PEG350)₄ | 7614 | 11471 | 1.51 | 41% |
| 2 | 4 | P(LA₇₅CL₂₅(500)-HDI-PEG550)₄ | 10681 | 17767 | 1.66 | 52% |
| 3 | 4 | P(LA₇₅CL₂₅(750)-HDI-PEG350)₄ | 14069 | 33264 | 2.36 | 42% |
| 4 | 4 | P(LA₇₅CL₂₅(750)-HDI-PEG550)₄ | 15991 | 33991 | 2.12 | 32% |
| 5 | 4 | P(LA₇₅CL₂₅(1000)-HDI-PEG550)₄ | 13348 | 20471 | 1.53 | 35% |
| 6 | 4 | P(LA₇₅CL₂₅(1000)-HDI-PEG750)₄ | 13898 | 19702 | 1.42 | 43% |
| 7 | 4 | P(LA₇₅CL₂₅(500)-HDI-PEG350)₄ | 7870 | 11836 | 1.50 | 41% |
| | | (large scale) | | | | |
| 8 | 4 | P(LA₇₅CL₂₅(500)-HDI-PEG550)₄ | 8253 | 10385 | 1.25 | 52% |
| 9 | 4 | P(LA₇₅CL₂₅(750)-HDI-PEG550)₄ | 10600 | 14860 | 1.40 | 42% |
| 10 | 4 | P(LA₇₅CL₂₅(1000)-HDI-PEG550)₄ | 12236 | 17433 | 1.42 | 35% |
| 11 | 4 | P(LA₇₅CL₂₅(2500)-HDI-PEG750)₄ | 18659 | 25299 | 1.35 | 23% |
| 12 | 4 | P(LA₇₅CL₂₅(2500)-HDI-PEG1000)₄ | 21210 | 27944 | 1.31 | 29% |
| 13 | 4 | P(LA₇₅CL₂₅(2500)-HDI-PEG2000)₄ | 23572 | 27764 | 1.17 | 44% |
| 14 | 4 | P(LA₇₅CL₂₅(500)-HDI-PEG350)₄ | 8645 | 13454 | 1.55 | 41% |
| 15 | 4 | P(LA₇₅CL₂₅(500)-HDI-PEG350)₄ | 8306 | 12467 | 1.50 | 41% |
| | | (repeat) | | | | |
| 16 | 4 | P(LA₇₅CL₂₅(500)-HDI-PEG350)₄ | 8355 | 12602 | 1.50 | 41% |
| | | (repeat) | | | | |
| 17 | 4 | P(LA₇₅CL₂₅(500)-HDI- | 8715 | 13264 | 1.52 | 41% |
| | | PEGMethylAcrylate350)₄ | | | | |
| 18 | 6 | P(LA₇₅CL₂₅(640)-HDI-PEG350)₆ | 12809 | 21200 | 1.65 | 35% |
| 19 | 6 | P(LA₁₀₀(265)-HDI-PEG350)₆ | 5641 | 13225 | 2.30 | 57% |
| 20 | 6 | P(LA₁₀₀(265)-HDI-PEG550)₆ | 6009 | 13849 | 2.30 | 67% |
| 21 | 8 | P(LA₁₀₀(265)-HDI-PEG350)₈ | 4774 | 86541 | 18.13 | 57% |
| 22 | 8 | P(LA₁₀₀(265)-HDI-PEG550)₈ | 6309 | 15246 | 2.41 | 67% |

### Storage Modulus:

The storage modulus (G′) and the loss modulus (G″) of the TRPs are measured using a rheometer. Dynamic rheology can measure these characteristics that represent the elastic contribution (storage modulus) to the viscoelastic fluid and the viscous contribution (loss modulus) to the stress applied. These modulii are measured by placing a sample in between two parallel plates under an oscillating shear stress and the resulting reaction force measured. Typically a cone and a plate rheometer is used for gels. The gel is placed on the cone and the bottom plate oscillated and torsion and the degree of twist measured. The shear stress is then calculated.

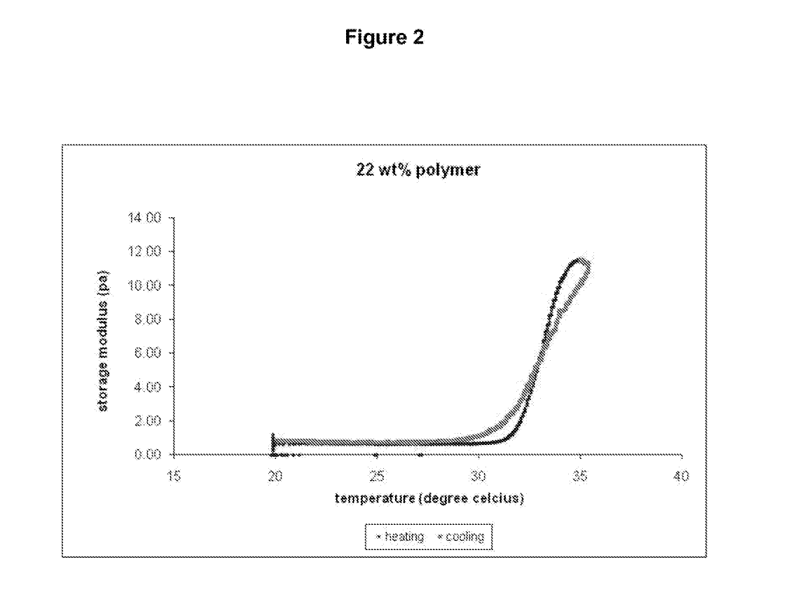

FIG. 2 is a graph showing the correlation between temperature and modulus for a composition containing a star block copolymer of the invention, (P(LA₇₅CL₂₅-HDI-PEG(550))₄), in which the storage modulus increases during gel formation (sol-gel) as temperature increases to physiological temperature, and the reversibility of the gel back to sol as the temperature is reduced. The graph illustrates the thermoreversible nature of the star block copolymer.

E. Star Block Copolymers with PEG Blocks and Different Hydrophobic Block Compositions

A range of star block copolymers were prepared without a linking group between the hydrophilic and hydrophobic polymer blocks. Using the general procedures described above by coupling the PEG block directly to the hydrophobic block (i.e. without Procedure B). The resulting star block copolymers are shown in Table 3.

TABLE 3

| | | | | LCST |
|---|---|---|---|---|
| Example | Polymer | Mn | P | (° C.) |
| 23 | PE-PLA 2000-PEG | 13532 | 2.05 | 44 |
| | 550 (PG7 55) | | | |
| 24 | PE-PLA-PCL 2000 (75:25)-PEG | 14018 | 2.27 | 39 |
| | 550 (PG5 95) | | | |
| 25 | PE-PLA-PCL 2000 (50:50)-PEG | 16205 | 1.83 | 42 |
| | 550 (PG7 34) | | | |
| 26 | PE-PLGA 5400 (75:25)-PEG | 8817 | 1.2 | 37 |
| | 550 (PG7 28) | | | |
| Comparative | PEG-PLGA(2800)-PEG | 4058 | 1.2 | 32 |
| Example 1 | (550) (linear) | | | |

The temperature (LCST) at which the polymer undergoes the sol to gel transformation indicates that the star copolymer remains a liquid at a higher temperature than the linear equivalent (Comparative Example 1), and does not gel until physiological temperature is reached (>36° C.). This suggests that the non-linear polymer architectures are ideal to act as a depot for drug delivery after injection into a human tissue.

### F. Typical Procedure for the Preparation of Aqueous Injectable Compositions:

Polymer solutions for release of lysozyme were prepared by dissolving purified polymer (0.582 g) initially in 1 g of distilled water. Dissolution of the polymer was carried out by placing the sample in the fridge (5-10° C.) over night with constant mixing using a vortex mixer. Lysozyme (60 mg) from chicken egg white (L6876-1G, Sigma-Aldrich) was separately dissolved in 0.358 g of distilled water and added to the completely dissolved polymer solution, mixed in using a fine spatula and finally with the vortex mixer to afford the final sample. A 0.5 g of the sample-lysozyme mixture amounting to 30% polymer in solution and 3% lysozyme concentration by weight was used in each experiment.

### Temperature Treatment of Polymer and Lysozyme Solution

In some long term release studies the polymer solutions containing the enzyme were initially subjected to heat-cool cycles and mixing the polymer thoroughly with purified water (a conditioning process). On each day the aqueous polymer solution containing the lysozyme was removed from the fridge mixed using a fine spatula and subsequently with a vortex mixer. It was then heated to 37° C. for 5-10 minutes and placed in the fridge. This process was carried out for 5 days prior to commencing the release study. This process was followed to promote increased chain entanglements between the polymer chains, its micelles and the polymer gel matrix formed thereafter.

### G. Typical Procedure for the Preparation of Enzyme Solution for Release Study:

The release study results were obtained in triplicate for each polymer sample, together with the necessary controls. A sample containing polymer and water without any lysozyme was utilized for each polymer as a blank to establish the baseline. Each sample in triplicate contained 0.5 g of the aqueous polymer-lysozyme mixture which was dispensed to the bottom of a small glass vial, incubated to 37° C. for 30 minutes prior to adding 2 mL of 1× phosphate buffer solution (PBS) maintained at the same temperature. All samples were kept at 37° C. in an incubator oven with gentle shaking (50 rpm) during the experiment. Aliquots of 1 mL from the PBS buffer solution were removed for analysis from each sample at each time point and replaced with fresh 1 mL of PBS at 37° C. Release medium was analyzed at each time point (10 mins, 24 hrs, 48 hrs, 5 days, 7 days, and 14 days) for release lysozyme. Both total soluble and enzymatically active lysozyme released from the polymer sample was measured using commercially available kits Bicinchoninic acid (BCA) protein assay kit from Sigma-Aldrich and EnzCheck lysozyme assay kit from Invitrogen respectively. Fresh calibration curves were constructed for each time point within the required range to ensure the correct measurement of the lysozyme released in each experimental sample.

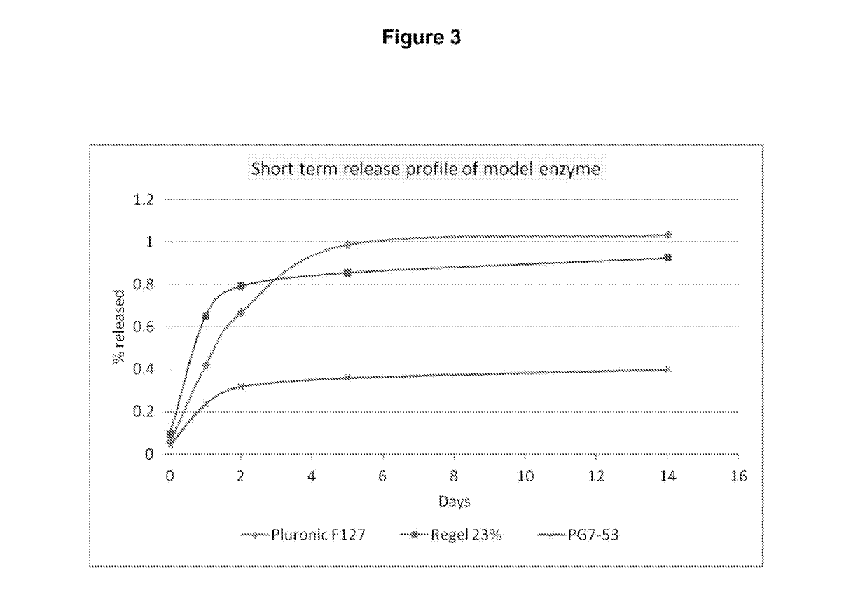

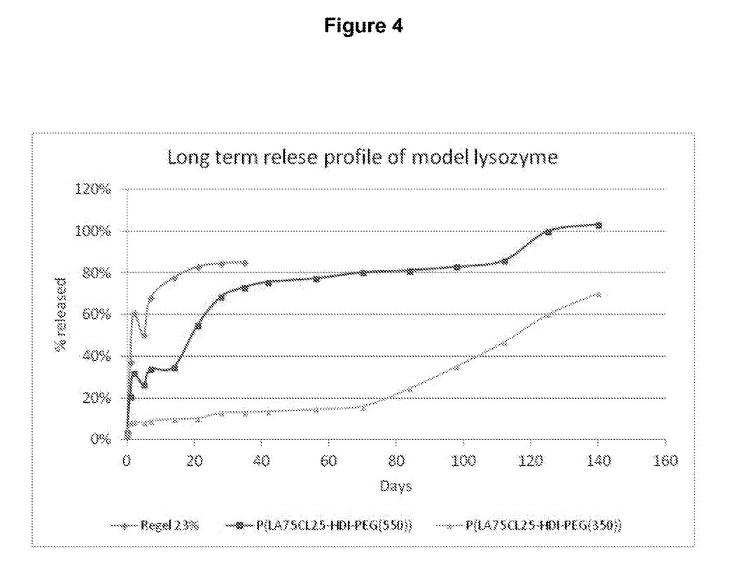

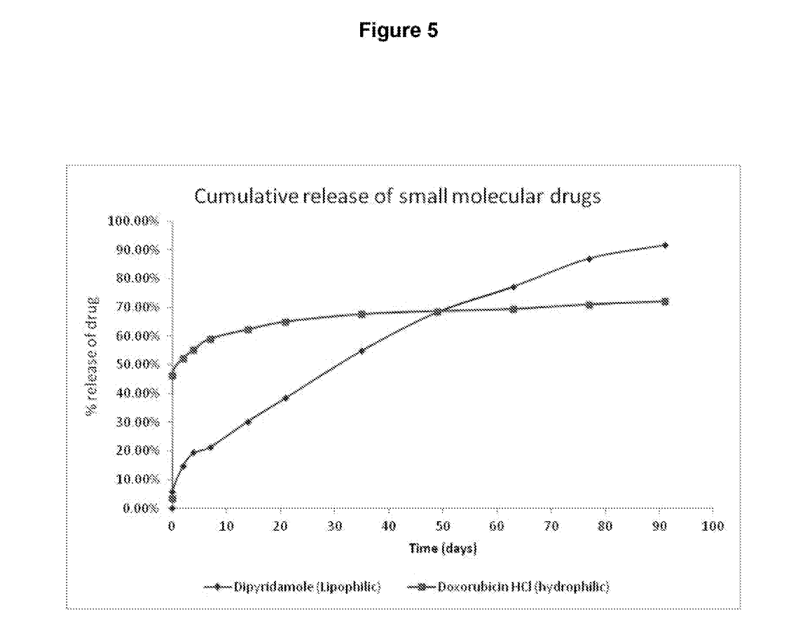

FIGS. 3 to 5 demonstrate some of the release profiles of this protein from compositions containing (P(LA₇₅CL₂₅-HDI-PEG(550)) 4) made according to the above methods compared to compositions containing prior art linear polymers Pluronic F127 and ReGel®.

### H. Typical Procedure for the Preparation of Small Molecule Drug Solution for Release Study:

Polymer Preparation with Dipyridamole (DP)

Dipyridamole (Sigma-Aldrich) 20 mg was dissolved in 2 mL of Dichloromethane (DCM—Merck) added to purified polymer (0.6 g) in a small round bottom flask. The polymer drug completely dissolves in dichloromethane. The DCM is then completely removed from the homogeneous mixture on a rotary evaporator and any residual DCM using a high vacuum pump. MilliQ water (1.4 g) of is then added to the polymer/DP mixture, mixed well at 5-10° C. to aid the dissolution, allowed to hydrate overnight.

The medium for the dipyridamole release study was prepared by the addition 2.4 g of Tween80 (Sigma-Aldrich) and 4.0 g of Cremophor EL (Sigma-Aldrich) to 93.6 g of 1×PBS (7.4 pH) to afford 100 g of the PBS solution.

Polymer Preparation with Doxorubicin (DR)

Doxorubicin (local collaborator) 20 mg was dissolved in 2 mL of Dichloromethane (DCM,—Merck) added to purified polymer (0.6 g) in a small round bottom flask. The polymer drug completely dissolves in dichloromethane. The DCM is then completely removed from the homogeneous mixture on a rotary evaporator and any residual DCM using a high vacuum pump. MilliQ water (1.4 g) of is then added to the polymer/DP mixture, mixed well at 5-10° C. to aid the dissolution, allowed to hydrate overnight.

### Release Study for Dipyridamole (DP)

The release study results were obtained in triplicate for each polymer sample, together with the necessary controls. A sample containing polymer and water without any drug was utilized for each polymer as a control to establish the baseline. Each sample in triplicate contained 0.5 g of the aqueous polymer-dipyridamole mixture which was dispensed to the bottom of a small glass vial, incubated to 37° C. for 30 minutes with general shaking (50 rpm) prior to adding 10 mL of 1× phosphate buffer solution (PBS) maintained at the same temperature. All of the release study samples were kept at 37° C. in an incubator oven with gentle shaking (50 rpm) during the experiment. Aliquots of 5 mL from the release medium were removed for analysis from each sample at each time point and the samples topped up with fresh 5 mL of PBS at 37° C. The aliquots were analysed at each time point (10 mins, 24 hrs, 48 hrs, 7, 14, 35 49, 63, 77 and 91 days) for the release doxorubicin. The total soluble DP released from the polymer sample in extraction medium was quantified against a calibration curve generated for known concentration of each drug at each sampling time. The absorbance at λ=408 nm for DP was measured for 1.0, 0.5, 0.25, 0.125 and 0.0625 mg/mL concentrations by using Varian 50 Bio UV-Visible spectrophotometer.

### Release Study for Doxorubicin (DR)

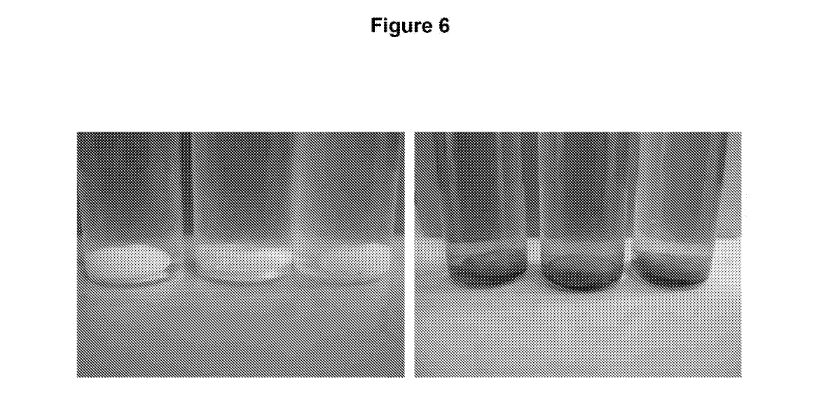

The release study results were obtained in triplicate for each polymer sample, together with the necessary controls. A sample containing polymer and water without any drug was utilized for each polymer as a control to establish the baseline. Each sample in triplicate contained 0.5 g of the aqueous polymer-doxorubicin mixture which was dispensed to the bottom of a small glass vial, incubated to 37° C. for 30 minutes with general shaking (50 rpm) prior to adding 10 mL of 1× phosphate buffer solution (PBS) maintained at the same temperature. All of the release study samples were kept at 37° C. in an incubator oven with gentle shaking (50 rpm) during the experiment. Aliquots of 5 mL from the release medium were removed for analysis from each sample at each time point and the samples topped up with fresh 5 mL of PBS at 37° C. The aliquots were analysed at each time point (10 mins, 24 hrs, 48 hrs, 7, 14, 35 49, 63, 77 and 91 days) for the release doxorubicin. The total soluble DR released from the polymer sample in extraction medium was quantified against a calibration curve generated for known concentration of each drug at each sampling time. The absorbance at λ=479 nm for DR was measured for 1.0, 0.5, 0.25, 0.125 and 0.0625 mg/mL concentrations by using Varian 50 Bio UV-Visible spectrophotometer. The results are illustrated in FIG. 6.

It is understood that various other modifications and/or alterations may be made without departing from the spirit of the present invention as outlined herein.

1. An injectable composition for controlled delivery of a bioactive agent comprising:
a biodegradable, thermoreversible, star block copolymer comprising a multi-valent central core and a plurality of biodegradable polymer arms attached to and extending from the central core;
an aqueous solvent; and
a bioactive agent,
wherein the composition is injectable as a liquid and the liquid is converted to a gel at physiological temperature and wherein the gelled composition provides release of an effective amount of the bioactive agent over a period of at least 7 days.

2. An injectable composition according to claim 1, wherein the gelled composition provides release of an effective amount of the bioactive agent over a period of at least 14 days.

3. An injectable composition according to claim 1, wherein the gelled composition provides release of an effective amount of the bioactive agent over a period of at least 28 days.

4. An injectable composition according to claim 1, wherein the star block copolymer is of formula A(BC)ₙ, wherein A represents a n-valent core and one of B and C represents a hydrophobic block and the other of B and C represents a hydrophilic block and n is an integer and is at least 3.

5. An injectable composition according to claim 4, wherein the weight ratio of B to C is in the range of from 10:1 to 1:10.

6. An injectable composition according to claim 4, wherein B represents a hydrophobic block and C represents a hydrophilic block.

7. An injectable composition according to claim 6, wherein the hydrophobic block has a molecular weight in the range of from about 500 to about 15,000.

8. An injectable composition according to claim 6, wherein B comprises a biodegradable polyester.

9. An injectable composition according to claim 8, wherein the polyester is formed from at least one monomer selected from the group consisting of D,L-lactide, D-lactide, L-lactide, D,L-lactic acid, D-lactic acid, L-lactic acid, glycolide, glycolic acid, ε-caprolactone, ε-hydroxy hexonoic acid, γ-butyrolactone, γ-hydroxy butyric acid, δ-valerolactone, δ-hydroxy valeric acid, hydroxy butyric acids, malic acid, mandelic acid and mixtures thereof.

10. An injectable composition according to claim 9, wherein the polyester is poly(lactic acid-co-caprolactone), wherein the mole ratio of lactic acid to caprolactone is in the range of from 90:10 to 10:90.

11. An injectable composition according to claim 6, wherein C comprises a hydrophilic polymer having a molecular weight in the range of from about 100 to about 3000.

12. An injectable composition according to claim 6, wherein C comprises a polyether.

13. An injectable composition according to claim 12 wherein the polyether is selected from the group consisting of poly(ethylene glycol), poly(propylene glycol), and copolymers thereof.

14. An injectable composition according to claim 4, wherein B and C are covalently coupled via a linking group.

15. An injectable composition according to claim 14, wherein the linking group is derived from a diisocyanate.

16. An injectable composition according to claim 4, wherein the star block copolymer comprises a further block (D) and is of formula A(BCD)ₙ.

17. An injectable composition according to claim 4, wherein n is an integer in the range of from 4 to 8.

18. An injectable composition according to claim 1, wherein the composition comprises no more than about 50% (w/w) of non-linear block copolymer.

19. An injectable composition according to claim 1, wherein the composition comprises at least 50% (v/w) of aqueous solvent.

20. An injectable composition according to claim 1, wherein the composition has a storage modulus in the range of 1 to 20 Pa at physiological temperature when in liquid form and when in gel form.

21. An injectable composition according to claim 1, wherein the bioactive agent is selected from the group consisting of hydrophilic drugs, hydrophobic drugs, protein drugs, hormones, genes or nucleic acids, oligonucleotides, polysaccharides and other sugars, lipids, gangliosides, vasoactive agents, neuroactive agents, anticoagulents, immunomodulating agents, anti-cancer agents, anti-inflammatory agents, antibiotics, antivirals, antisense, antigens and antibodies.

22. An injectable composition according to claim 1, further comprising an additive that enhances control of the release of the bioactive agent.

23. An injectable composition according to claim 22, wherein the additive is a polysaccharide.

24. An injectable composition according to claim 1, wherein release of the bioactive agent from the gelled composition occurs over at least two separate phases.

25. An injectable composition according to claim 1, wherein no more than about 30% of the bioactive agent is released from the gelled composition within the first 24 hours after gel formation.

26-29. (canceled)
